(19) United States
(12) Patent Application Publication
(10) Pub. No.: US 2015/0209343 A1
(43) Pub. Date: Jul. 30, 2015

(54) TOPICAL DERMAL COMPOSITIONS

(71) Applicant: Allergan, Inc., Irvine, CA (US)

(72) Inventors: Guang Wei Lu, Irvine, CA (US); Fangjing Wang, Irvine, CA (US); Kevin S. Warner, Anaheim, CA (US); Rong Yang, Mission Viejo, CA (US); Robert H. Mo, Princeton, NJ (US)

(21) Appl. No.: 14/607,531

(22) Filed: Jan. 28, 2015

Related U.S. Application Data
(60) Provisional application No. 61/932,584, filed on Jan. 28, 2014.

(51) Int. Cl. A61K 31/4436; A61K 9/10; A61K 9/00; A61K 9/51

(52) U.S. Cl. CPC A61K 31/4436; A61K 9/5123; A61K 9/10; A61K 9/0014; A61K 9/5192

(57) ABSTRACT

The present invention provides topical dermal compositions useful for treating a variety of conditions associated with excess sebum production, such as for example acne.

### CROSS-REFERENCE TO RELATED APPLICATIONS

This application is a non-provisional application which claims the benefit of U.S. provisional application 61/932,584 entitled “Topical Dermal Compositions” filed on Jan. 28, 2014 with docket number 19349PROV (AP) which is incorporated herein by reference in its entirety and serves as the basis for a benefit and/or priority claim of the present application.

### FIELD

The present invention relates generally to topical dermal compositions for treating (both therapeutically treating and cosmetically treating) a number of different dermatological (that is skin related) diseases and conditions (such as for example acne). In particular the present invention relates to topically administered dermal compositions which comprise a retinoid encapsulated (or synonymously encompassed or enclosed or entrapped) by a lipid (such as a lipid which is a sold at a room temperature of from about 20° C. to about 25° C. and at about one atmospheric pressure, that is at sea level). The solid lipid (encapsulating a retinoid) can be in the form of “nanoparticles” which have a diameter of from about one tenth of a micron to about ten microns. More particularly the present invention relates to topical dermal compositions comprising a retinoid encapsulated by solid lipid nanoparticles that are mixed into or dispersed within a gel, the gel serving as a vehicle for the retinoid encapsulating solid lipid nanoparticles.

### BACKGROUND

Human skin is composed of three primary layers, the stratum corneum, the epidermis, and the dermis. The outer layer is the stratum corneum. Its primary function is to serve as a barrier to the external environment. Lipids are secreted to the surface of the stratum corneum, where they decrease the stratum corneum's water permeability. Sebum typically constitutes 95% of these lipids. See e.g., Sebum, Cosmetics, and Skin Care by Abramovits W., et al., in Dermatologic Clinics, volume 18, issue 4, pages 617-620 (2000). In addition to maintaining the epidermal permeability barrier, sebum transports anti-oxidants to the surface of the skin and protects against microbial colonization

Sebum is produced in the sebaceous glands. These glands are present over most of the surface of the body. The highest concentration of these glands occurs on the scalp, the forehead, and the face. Despite the important physiological role that sebum plays, many individuals experience excess sebum production, especially in the facial area. An increased rate of sebum excretion is termed seborrhea.

Seborrhoeic dermatitis is also associated with seborrhea. The condition is characterized by the appearance of red, flaking, greasy areas of skin, most commonly on the scalp, nasolabial folds, ears, eyebrows and chest. In the clinical literature seborrhoeic dermatitis may be also referred to as “sebopsoriasis,” “seborrhoeic eczema,” “dandruff,” and “pityriasis capitis.” Yeast infections are a causative factor in seborrhoeic dermatitis. The yeast thrives on sebum and leaves high concentrations of unsaturated fatty acids on the skin, thereby irritating it.

Acne vulgaris is associated with clinical seborrhea and there is a direct relationship between the sebum excretion rate and the severity of acne vulgaris. Although sebum production increases during adolescence (particularly in boys, because of androgen stimulation), increased sebum alone does not cause acne. Bacteria, most importantly P. acnes, feed on sebum and as a result are present in increased numbers in persons who have acne. Much of the inflammation associated with acne arises from the action of enzymes produced by the bacteria.

Acne vulgaris is characterized by areas of skin with seborrhea (scaly red skin), comedones (blackheads and whiteheads), papules (pinheads), pustules (pimples), nodules (large papules), and in more severe cases, scarring. It mostly affects skin with the densest population of sebaceous follicles, such as the face, upper chest, and back.

There are four key pathogenic factors of acne:
follicular hyperkeratinization
propionibacterium acnes (P. acnes)
inflammation
excessive sebum production (seborrhea)

Acne is still a very underserved market with treatment options that are only marginally effective. Only one product, oral Accutane® (isotretinoin) that reduces sebum production has been highly effective, but at the expense of a black box warning with significant side effects including teratogenicity that require extensive patient monitoring. Accutane® is indicated only for acne which is severe and recalcitrant to other treatment

Topical therapy is often preferred over oral therapy because of the reduced risk for adverse systemic effects. The most common topical drugs for acne can be divided into the following categories:
Retinoids (i.e., tazarotene, tretinoin, adapalene)
Antibiotics (i.e., clindamycin)
Benzoyl peroxide (BPO)
Others (i.e., dapsone, azelaic acid)

While many topical therapies are available, none of them address all four factors and most specialize in a few of these factors. Currently, no topical therapies in the market address excessive sebum production. Sebum is produced by the sebaceous gland, which is an appendage of the hair follicle, so it makes sense to target the sebaceous gland for more effective therapy. Since P. acnes depends on sebum to live, reduction of sebum is also thought to indirectly reduce P. acnes.

Topical retinoids primarily act by normalizing infundibular hyperkeratinization and reducing inflammation, hence topical retinoids remain a mainstay for treatment of mild-to-moderate acne. The current topical retinoid formulations do not inhibit sebum production and their use is often limited by local tolerability (i.e., skin irritation).

The following publications may be relevant to the present invention: Solid lipid nanoparticles (SLN) for controlled drug delivery, a review of the state of the art, by Muller, R., et al., European Journal of Pharmaceutics and Biopharmaceutics 50 (2000) 161-177; Lipid nanoparticles for improved topical application of drugs for skin diseases by Schäfer-Korting, M., et al., in Advanced Drug Delivery Reviews 59 (2007) 427-443; Castro, G., et al., Formation of ion pairing as an alternative to improve encapsulation and stability and to reduce skin irritation of retinoic acid loaded in solid lipid nanoparticles, International Journal of Pharmaceutics 381 (2009) 77-83; Castro G., et al., Comedolytic effect and reduced skin irritation of a new formulation of all-trans retinoic acid-loaded solid lipid nanoparticles for topical treatment of acne, Arch Dermatol Res (2011); 303:513-520; U.S. Pat. No. 5,851,538; international patent application publication WO 2004/082666; Canadian patent 2 519 697; US published patent application US 2006/0257334, and; US published patent application US 2012/0328670.

Additionally, the company SkinMedica (Carlsbad, Calif.) sells a topically applied product called “Retinol Complex” for enhancing skin tone which comprises lipid particles encapsulating retinol, as well as water, cetyl ethylhexanoate, glycine soja oil, niacinamide, polyacrylate-13, caprylic/capric triglyceride, squalane, palmitoyl tripeptide-8, dunaliella salina extract, magnolia grandiflora bark extract, tocopherol, tocotrienols, ceramide 3, bisabolol, phytosterols, squalene, tocopheryl acetate, oryza sativa bran cera, glycerin, polysorbate 20, butylene glycol, cetyl palmitate, laureth-23, trideceth-6 phosphate, sodium hydroxide, dicaprylyl ether, lauryl alcohol, polyisobutene, dextran, potassium sorbate, disodium EDTA, phenoxyethanol, and ethylhexylglycerin.

Thus, there is a need in the art, therefore, for topical compositions capable of reducing sebum production and treating the conditions associated with it.

### SUMMARY

The present invention satisfies this need by providing topical dermal compositions which can comprise microspheres, microparticles and/or nanoparticles (including nanoparticles made of a lipid or lipids) which encompass an active ingredient (such as a retinoid). The retinoid encompassing microspheres, microparticles and/or nanoparticles are mixed with, dispersed within, suspended by, emulsified with or by, etc, a carrier or vehicle, which carrier or vehicle can be gel, lotion, cream or the like. A preferred embodiment of the present invention comprises solid lipid nanoparticles encapsulating a retinoid and present within in a semi-solid (i.e. gel) vehicle.

The active ingredient can be a retinoid (such as tazarotene or isotretinoin), a prostaglandin or a prostamide. The retinoid is preferably tazarotene or a salt, ester, or amide thereof. The dermally applied compositions of the present invention are useful for treating a variety of dermatological conditions, for example dermatological conditions associated with excess sebum production, such as for example acne.

By employing a dermal composition of the present invention a retinoid (such as tazarotene) can be targeted for and delivered deep into the skin of a patient into the hair follicles of the patient where the retinoid reaches the sebaceous glands and significantly inhibit production of sebum by the sebaceous glands, thereby providing an effective treatment of a dermatological condition such as acne. The present dermal compositions can provide sustained or extended delivery of the retinoid from the encapsulating lipoid nanoparticle over a period of time from as little as a few seconds (i.e. over about 5 second after dermal application of the dermal composition to over about 100 seconds), to a few minutes (i.e. over about 1 minute after dermal administration to about 15 minutes), to a few days (i.e. from about 1 day after dermal administration to about 3 days), to several weeks (i.e. from about 1 week after dermal administration to about 3 weeks), wherein the retinoid is released from the lipid nanoparticles under either first order or under zero order release rate kinetics.

Importantly, the compositions of the invention result in fewer and reduced side effects to the surface of the skin in comparison to other known retinoid creams, gels or other formulations, which other inferior formulations do not provide a sustained or extended release formulation for the topical treatment of acne.

One embodiment of the present invention comprises a topical dermal composition which includes a plurality of nanoparticles, wherein the nanoparticles comprise a biodegradable lipid, and a retinoid or a pharmaceutically acceptable salt, ester, or amide thereof, a population of the nanoparticles have an average diameter between about 0.1 μm and about 10 μm.

The present invention also includes methods for treating a condition associated with excess sebum production. Such methods can be performed, for example, by topically applying to the skin of a patient in need thereof a composition within the scope of the present invention.

Thus, the present invention encompasses a composition for topical dermal administration comprising a retinoid encompassed by lipid particles in a gel vehicle. The retinoid can be tazarotene, the lipid is preferably a solid at room temperature and the lipid has a melting point at about or greater than about 32° C. “Room temperature” means 20-25 degrees C., and preferably about 23 degrees C. The composition can have the retinoid encompassed by a plurality of biodegradable lipid nanoparticles that are solid at room temperature. The composition can comprise a surfactant and the solid lipid nanoparticles can have an average diameter no greater than about 10 microns, no greater than about 5 μm, no greater than about 3 μm or no greater than about 1 μm. In the composition the lipid can be selected from the group consisting of glyceryl dibehenate, glyceryl behenate myristyl myristate, myristyl laurate, triglycerides of C₁₀-C₂₂ fatty acids, propylene glycol monopalmiteostearate, cetyl palmitate, isostearyl isostearate, and propylene glycol monopalmiteostearate, and combinations thereof. The invention also encompasses a method for treating a condition associated with excess sebum production by topically applying to the skin of a patient in need thereof the composition set forth in this paragraph and the condition treated can be for example acne vulgaris, seborrhoeic dermatitis, psoriasis, or keratosis pilaris.

The invention also encompasses a composition for topical dermal administration comprising a retinoid encapsulated by or encompassed by a lipid. The retinoid can be tazarotene and preferably the lipid is solid at room temperature and the lipid has a melting point between bout 32° C. to about 37° C. The lipid is formed into a plurality of biodegradable, solid lipid nanoparticles encompassing or encapsulating the tazarotene, which formulation (tazarotene encapsulated by solid lipid nanoparticles) can be abbreviated as “TazSLN”. Importantly, the TazSLN is mixed with or dispersed within (preferably) a gel as a carrier or as a vehicle for the TazSLN, thereby providing a dermal composition which can be abbreviated as “TazSLG” (note that TazSLN in a gel vehicle forms the TazSLG). The vehicle or carrier does not comprise a lipid. Preferably the lipid which comprises the solid lipid nanoparticles is selected from the group consisting of glyceryl dibehenate, glyceryl behenate myristyl myristate, myristyl laurate, triglycerides of C₁₀-C₂₂ fatty acids, propylene glycol monopalmiteostearate, cetyl palmitate, isostearyl isostearate, and propylene glycol monopalmiteostearate, and combinations thereof. Significantly, TazSLG dermal compositions described herein are stable for at least six months when stored at room temperature. Such stability being demonstrated infra by both little or no separation (i.e. no precipitation out) of the TazSLN out of the TazSLG dermal composition, and as well by continued excellent follicular delivery of the tazarotene from the six month or longer room temperature stored and then dermally applied TazSLG, with reduced skin irritation (as compared to the amount of skin irritation which results from dermal application of a same strength composition which comprises the same concentration of tazarotene in a similar vehicle, such as a gel vehicle, such as Tazaroc Gel—i.e. a control formulation), and as well as by an efficacy in the reduction of sebum and acne treatment which is greater than that obtained by the control formulation.

The invention also encompasses a composition for topical dermal administration comprising: (a) tazarotene; (b) a plurality of biodegradable, solid lipid nanoparticles encompassing or encapsulating the tazarotene, thereby forming TazSLN; and (c) a gel acting as a carrier or as a vehicle for the TazSLN, thereby forming TazSLG. The lipid can be selected from the group consisting of glyceryl dibehenate, glyceryl behenate myristyl myristate, myristyl laurate, triglycerides of C₁₀-C₁₈ fatty acids, propylene glycol monopalmiteostearate, cetyl palmitate, isostearyl isostearate, and propylene glycol monopalmiteostearate, and combinations thereof.

Additionally, the invention encompasses a method for treating a condition associated with excess sebum production, the method comprising the step of topically applying to the skin of a patient in need of such treatment a dermal composition comprising:
(1) solid lipid nanoparticles, wherein the solid lipid nanoparticles comprise
   a) a lipid from the group consisting of glyceryl dibehenate, glyceryl behenate myristyl myristate, myristyl laurate, triglycerides of C₁₀-C₂₂ fatty acids, propylene glycol monopalmiteostearate, cetyl palmitate, isostearyl isostearate, and propylene glycol monopalmiteostearate, and combinations thereof.
   biodegradable polymer, and
   b) encapsulated by or encompassed by the lipid a compound of the formula:

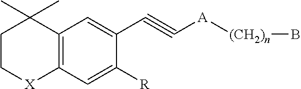

wherein:
X is S, O, or —N(R¹)— where R¹ is hydrogen or lower alkyl;
R is hydrogen or lower alkyl;
A is pyridinyl, thienyl, furyl, pyridazinyl, pyrimidinyl or pyrazinyl;
n is 0-2;
B is selected from the group consisting of: H, —COOH or a pharmaceutically acceptable salt, ester or amide of said —COOH group, —CH₂OH or an ether or ester derivative of said —CH₂OH group, —CHO or an acetal derivative of said —CHO group, and —COR² or a ketal derivative of said —COR² group, wherein R² is —(CH₂)ₘCH₃ wherein m is 0-4;
and;
(2) a gel carrier or vehicle for the solid lipid nanoparticles,
   wherein the solid lipid nanoparticles have an average diameter between about 0.1 μm and about 10 μm; and wherein the compound penetrates the hair follicle to the depth of the sebaceous gland, and acts directly on the gland to reduce sebum production by the gland.

Additionally, the invention encompasses a method for treating a condition associated with excess sebum production, the method comprising the step of topically applying to the skin of a patient in need of such treatment a dermal composition comprising:
(1) solid lipid nanoparticles, wherein the solid lipid nanoparticles comprise:
   a) a lipid from the group consisting of glyceryl dibehenate, glyceryl behenate myristyl myristate, myristyl laurate, triglycerides of C₁₀-C₁₈ fatty acids, propylene glycol monopalmiteostearate, cetyl palmitate, isostearyl isostearate, and propylene glycol monopalmiteostearate, and combinations thereof.
   biodegradable polymer, and
   b) encapsulated by or encompassed by the first lipid a compound of the formula:

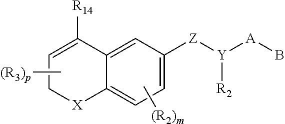

or a pharmaceutically acceptable salt thereof, wherein:
   X is S, O, NR′ where R′ is H or alkyl of 1 to 6 carbons, or X is [C(R₁)₂]ₙ where R₁ is independently H or alkyl of 1 to 6 carbons, and n is an integer between, and including, 0 and 2, and;
   R₂ is hydrogen, lower alkyl of 1 to 6 carbons, F, Cl, Br, I, CF₃, fluoro substituted alkyl of 1 to 6 carbons, OH, SH, alkoxy of 1 to 6 carbons, or alkylthio of 1 to 6 carbons, and;
   R₃ is hydrogen, lower alkyl of 1 to 6 carbons or F, and;
   m is an integer having the value of 0-3, and;
   p is an integer having the value of 0-3, and;
   Z is —C≡C—, —N═N—, —N═CR₁—, —CR₁═N, —(CR₁═CR₁)_{n′}— where n′ is an integer having the value 0-5, —CO—NR₁—, —CS—NR₁—, —NR₁—CO, —NR₁—CS, —COO—, —OCO—, —CSO—, or —OCS—;
   Y is a phenyl or naphthyl group, or heteroaryl selected from a group consisting of pyridyl, thienyl, furyl, pyridazinyl, pyrimidinyl, pyrazinyl, thiazolyl, oxazolyl, imidazolyl and pyrrazolyl, said phenyl and heteroaryl groups being optionally substituted with one or two R₂ groups, or, when Z is —(CR₁═CR₁)_{n′}— and n′ is 3, 4 or 5 then Y represents a direct valence bond between said (CR₂═CR₂)_{n′} group and B;
   A is (CH₂)_{q} where q is 0-5, lower branched chain alkyl having 3-6 carbons, cycloalkyl having 3-6 carbons, alkenyl having 2-6 carbons and 1 or 2 double bonds, alkynyl having 2-6 carbons and 1 or 2 triple bonds;
   B is hydrogen, COOH or a pharmaceutically acceptable salt thereof, COOR₈, CONR₉R₁₀, —CH₂OH, CH₂OR₁₁, CH₂OCOR₁₁, CHO, CH(OR₁₂)₂, CHOR₁₃O, —COR₇, CR₇(OR₁₂)₂, CR₇OR₁₃O, or tri-lower alkylsilyl, where R₇ is an alkyl, cycloalkyl or alkenyl group containing 1 to 5 carbons, R₈ is an alkyl group of 1 to 10 carbons or trimethylsilylalkyl where the alkyl group has 1 to 10 carbons, or a cycloalkyl group of 5 to 10 carbons, or R₈ is phenyl or lower alkylphenyl, R₉ and R₁₀ independently are hydrogen, an alkyl group of 1 to 10 carbons, or a cycloalkyl group of 5-10 carbons, or phenyl or lower alkylphenyl, R₁₁ is lower alkyl, phenyl or lower alkylphenyl, R₁₂ is lower alkyl, and R₁₃ is divalent alkyl radical of 2-5 carbons, and
   R₁₄ is (R₁₅)ᵣ-phenyl, (R₁₅)ᵣ-naphthyl, or (R₁₅)ᵣ-heteroaryl where the heteroaryl group has 1 to 3 heteroatoms selected from the group consisting of O, S and N, r is an integer having the values of 0-5, and
   R₁₅ is independently H, F, Cl, Br, I, NO₂, N(R₈)₂, N(R₈)COR₈, NR₈CON(R₈)₂, OH, OCOR₈, OR₈, CN, an alkyl group having 1 to 10 carbons, fluoro substituted alkyl group having 1 to 10 carbons, an alkenyl group having 1 to 10 carbons and 1 to 3 double bonds, alkynyl group having 1 to 10 carbons and 1 to 3 triple bonds, or a trialkylsilyl or trialkylsilyloxy group where the alkyl groups independently have 1 to 6 carbons;
and;
(2) a gel as a carrier or as a vehicle for the solid lipid nanoparticles,
   wherein the solid lipid nanoparticles have an average diameter between about 0.1 μm and about 10 μm; and wherein the compound penetrates the hair follicle to the depth of the sebaceous gland, and acts directly on the gland to reduce sebum production by the gland.

The present invention includes a composition for topical dermal administration comprising tazarotene encapsulated by a lipid, and a gel vehicle into which the lipid encapsulated retinoid is mixed or dispersed. Preferably, the lipid is a solid at room temperature and more preferably the lipid has a melting point at about or greater than about 32° C. In the invention the lipid is in the form of a plurality of biodegradable, solid lipid nanoparticles encompassing or encapsulating the tazarotene, thereby providing TazSLN, and in turn TazSLN in the gel vehicle provides TazSLG. The composition can further comprising a surfactant and the gel is preferably a polymeric formed by use of a gelling agent. Preferably, the solid lipid nanoparticles have an average diameter (i.e. size as determined for example by light scattering) no greater than about 5 μm, such as an average diameter no greater than about 3 μm and an average diameter no greater than about 1 μm.

The lipid used in the invention is preferably selected from the group consisting of glyceryl dibehenate, glyceryl behenate myristyl myristate, myristyl laurate, triglycerides of C₁₀-C₂₂ fatty acids, propylene glycol monopalmiteostearate, cetyl palmitate, isostearyl isostearate, and propylene glycol monopalmiteostearate, and combinations thereof, more preferred lipids are myristyl myristate, glyceryl dibehenate or glyceryl dibehenate.

The invention includes a method for treating a condition associated with excess sebum production by applying to the skin of a patient in need thereof the dermal compositions set forth above and the condition treated can be for example acne vulgaris, seborrhoeic dermatitis, psoriasis, and keratosis pilaris.

A preferred composition within the scope of the invention can comprise tazarotene encapsulated by a plurality of biodegradable, solid lipid nanoparticles, thereby forming TazSLN, and a polymeric gel vehicle into which the TazSLN is mixed or dispersed, thereby forming TazSLG, wherein the lipid comprising solid the lipid nanoparticles is selected from the group consisting of glyceryl dibehenate, glyceryl behenate, myristyl myristate, myristyl laurate, triglycerides of C₁₀-C₂₂ fatty acids, propylene glycol monopalmiteostearate, cetyl palmitate, isostearyl isostearate, and propylene glycol monopalmiteostearate, and combinations thereof, and wherein the polymeric gel is comprises a gel or a gelling agent selected from the group consisting of a carbomer, acacia, alginic acid, bentonite, carboxymethylcellulose, ethylcellulose, gelatin, hydroxyethylcellulose, hydroxypropyl cellulose, magnesium aluminum silicate, methylcellulose, poloxamers, polyvinyl alcohol, sodium alginate, tragacanth, and xanthan gum.

Additionally, a further method within the scope of the invention is a method for treating a condition associated with sebum production, the method comprising the step of topically applying to the skin of a patient in need of such treatment a dermal composition comprising:
solid lipid nanoparticles, wherein the solid lipid nanoparticles comprise a lipid selected from the group consisting of glyceryl dibehenate, glyceryl behenate myristyl myristate, myristyl laurate, triglycerides of C₁₀-C₂₂ fatty acids, propylene glycol monopalmiteostearate, cetyl palmitate, isostearyl isostearate, and propylene glycol monopalmiteostearate, and combinations thereof, and encapsulating by or encompassed by all or substantially all of the solid lipid nanoparticles is a compound of the formula:

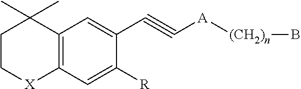

wherein:
   X is S, O, or —N(R¹)— where R¹ is hydrogen or lower alkyl;
   R is hydrogen or lower alkyl;
   A is pyridinyl, thienyl, furyl, pyridazinyl, pyrimidinyl or pyrazinyl;
   n is 0-2;
   B is selected from the group consisting of: H, —COOH or a pharmaceutically acceptable salt, ester or amide of said —COOH group, —CH₂OH or an ether or ester derivative of said —CH₂OH group, —CHO or an acetal derivative of said —CHO group, and —COR² or a ketal derivative of said —COR² group, wherein R² is —(CH₂)ₘCH₃ wherein m is 0-4;
and;
a polymeric gel as a carrier or as a vehicle for the solid lipid nanoparticles, wherein the solid lipid nanoparticles have an average diameter between about 0.1 μm and about 10 μm;
   and wherein the compound penetrates the hair follicle to the depth of the sebaceous gland, and acts directly on the gland to reduce sebum production by the sebaceous gland, thereby treating the condition.

Another method within the scope of the invention is a method for treating a condition associated with excess sebum production, the method comprising the step of topically applying to the skin of a patient in need of such treatment a dermal composition comprising:
(1) solid lipid nanoparticles, wherein the solid lipid nanoparticles comprise
   a) a lipid from the group consisting of glyceryl dibehenate, glyceryl behenate myristyl myristate, myristyl laurate, triglycerides of C₁₀-C₂₂ fatty acids, propylene glycol monopalmiteostearate, cetyl palmitate, isostearyl isostearate, and propylene glycol monopalmiteostearate, and combinations thereof, and;
   b) encapsulated by or encompassed by all or by significantly all of the solid lipid nanoparticles a compound of the formula:

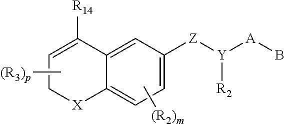

or a pharmaceutically acceptable salt thereof, wherein:
   X is S, O, NR′ where R′ is H or alkyl of 1 to 6 carbons, or X is [C(R₁)₂] where R₁ is independently H or alkyl of 1 to 6 carbons, and n is an integer between, and including, 0 and 2, and;
   R₂ is hydrogen, lower alkyl of 1 to 6 carbons, F, Cl, Br, I, CF₃, fluoro substituted alkyl of 1 to 6 carbons, OH, SH, alkoxy of 1 to 6 carbons, or alkylthio of 1 to 6 carbons, and;
   R₃ is hydrogen, lower alkyl of 1 to 6 carbons or F, and;
   m is an integer having the value of 0-3, and;
   p is an integer having the value of 0-3, and;
   Z is —C≡C—, —N═N—, —N═CR₁—, —CR₁═N, —(CR₁═CR₁)_{n′}— where n′ is an integer having the value 0-5, —CO—NR₁—, —CS—NR₁—, —NR₁—CO, —NR₁—CS, —COO—, —OCO—, —CSO—, or —OCS—;
   Y is a phenyl or naphthyl group, or heteroaryl selected from a group consisting of pyridyl, thienyl, furyl, pyridazinyl, pyrimidinyl, pyrazinyl, thiazolyl, oxazolyl, imidazolyl and pyrrazolyl, said phenyl and heteroaryl groups being optionally substituted with one or two R₂ groups, or, when Z is —(CR₁═CR₁)_{n′}— and n′ is 3, 4 or 5 then Y represents a direct valence bond between said (CR₂═CR₂)_{n′} group and B;
   A is (CH₂)_{q} where q is 0-5, lower branched chain alkyl having 3-6 carbons, cycloalkyl having 3-6 carbons, alkenyl having 2-6 carbons and 1 or 2 double bonds, alkynyl having 2-6 carbons and 1 or 2 triple bonds;
   B is hydrogen, COOH or a pharmaceutically acceptable salt thereof, COOR₈, CONR₉R₁₀, —CH₂OH, CH₂OR₁₁, CH₂OCOR₁₁, CHO, CH(OR₁₂)₂, CHOR₁₃O, —COR₇, CR₇(OR₁₂)₂, CR₇OR₁₃O, or tri-lower alkylsilyl, where R₇ is an alkyl, cycloalkyl or alkenyl group containing 1 to 5 carbons, R₈ is an alkyl group of 1 to 10 carbons or trimethylsilylalkyl where the alkyl group has 1 to 10 carbons, or a cycloalkyl group of 5 to 10 carbons, or R₈ is phenyl or lower alkylphenyl, R₉ and R₁₀ independently are hydrogen, an alkyl group of 1 to 10 carbons, or a cycloalkyl group of 5-10 carbons, or phenyl or lower alkylphenyl, R₁₁ is lower alkyl, phenyl or lower alkylphenyl, R₁₂ is lower alkyl, and R₁₃ is divalent alkyl radical of 2-5 carbons, and
   R₁₄ is (R₁₅)ᵣ-phenyl, (R₁₅)ᵣ-naphthyl, or (R₁₅)ᵣ-heteroaryl where the heteroaryl group has 1 to 3 heteroatoms selected from the group consisting of O, S and N, r is an integer having the values of 0-5, and
   R₁₅ is independently H, F, Cl, Br, I, NO₂, N(R₈)₂, N(R₈)COR₈, NR₈CON(R₈)₂, OH, OCOR₈, OR₈, CN, an alkyl group having 1 to 10 carbons, fluoro substituted alkyl group having 1 to 10 carbons, an alkenyl group having 1 to 10 carbons and 1 to 3 double bonds, alkynyl group having 1 to 10 carbons and 1 to 3 triple bonds, or a trialkylsilyl or trialkylsilyloxy group where the alkyl groups independently have 1 to 6 carbons;
and;
(2) a polymeric gel as a carrier or as a vehicle for the solid lipid nanoparticles,
   wherein the solid lipid nanoparticles have an average diameter between about 0.1 μm and about 10 μm; and wherein the compound penetrates the hair follicle to the depth of the sebaceous gland, and acts directly on the gland to reduce the excess sebum production by the gland, thereby treating the condition.

The invention also includes a process for making TazSLG, as by the following steps of:
(a) preparing a lipid phase by making a hot melt homogenization of a lipid by heating a mixture of a lipid and a retinoid to a temperature of between about 75° C. and about 90° C.;
(b) preparing an aqueous phase comprising water and a surfactant, also heated to a temperature of between about 75° C. and about 90° C.;
(c) mixing together the lipid phase and the aqueous phase;
(d) cooling (i.e. quenching) the step (c) mixture to a temperature between about 5° C. and about 15° C. (and preferably to a temperature between about 10° C. and about 15° C.) over a period of time of about 5 minutes, thereby preparing a suspension in water of tazarotene encapsulated solid lipid particles (TazSLN);
(e) preparing a gel phase by adding a carbomer gelling agent to water and mixing; and
(f) mixing together the gel phase and the TazSLN suspension, and adjust the pH of the mixture to between about pH 5.5 to about pH 6.0, thereby making TazSLG.

The solid lipid particles encapsulating tazarotene and in a gel vehicle (TazSLG) made by the process above are also part of the invention.

Furthermore the invention also encompasses a composition for topical dermal administration comprising a retinoid encapsulated by a fatty acid ester, and a gel vehicle into which the fatty acid ester encapsulated retinoid is mixed or dispersed.

An exemplary composition for topical dermal administration within the scope of the invention can comprise:

(a) a population of solid lipid nanoparticles comprising:
(i) butylated hydroxytolune;
(ii) butylated hydroxyanisole;
(iii) a surfactant;
(iv) tazarotene, and;
(v) glyceryl behenate, and;
   (b) a gel comprising:
(i) ethylenediaminetetraacetic acid;
(ii) a carboxypolymethylene carbomer homopolymer
(iii) sodium thiosulfate;
(iv) methylparaben;
(v) propylparaben;
(vi) phenoxyethanol, and;
(vii) sodium thiosulfate,
   wherein the solid lipid nanoparticles have with an average particle size of between about 1 micron and about 10 microns, the solid lipid nanoparticles encapsulate or encompass substantially all the tazarotene, and the solid lipid nanoparticles are mixed into or dispersed within the gel, the gel serving as a vehicle for the solid lipid nanoparticles.

### DRAWINGS

FIG. 1 shows a bar graph showing on the x axis the four Table 3 formulations 9, 10, 11 and 12 tested (each formulation being a tazarotene loaded SLN formulation with a D90 of about 1.9 microns), and on the y axis the area of the sebaceous glands on the drug (the selected tazarotene SLN formulation) treated side as a percent of the control (untreated side) sebaceous gland area, in the hamster flank model. A FIG. 1 y axis lower percentage value means a reduction of the drug treated sebaceous gland area as compared to the untreated (control) sebaceous gland area. It is known that a reduced sebaceous gland area directly corresponds to a reduced sebum production by the gland.

FIG. 2 shows a bar graph showing on the x axis the four Table 4 formulations 13, 14, 15 and 16 tested (each being a tazarotene loaded SLN formulation with a D90 of about 0.4 microns), and on the y axis the area of sebaceous glands on the drug treated side as a percent of the control untreated side gland area, in the hamster flank model. As in FIG. 1 the lower percentage means more reduction of sebaceous gland by drug treatment and reduced gland size means less sebum production.

FIG. 3 shows a bar graph showing on the x axis the three Table 5 formulations 17, 18, and 19 tested (each being an Accutane loaded SLN formulation with a D90 of about 3.06 microns), and on the y axis the area of sebaceous glands on the drug treated side as percent of the control untreated side gland area, in the hamster flank model. As in FIG. 1 the lower percentage means more reduction of sebaceous gland by drug treatment and reduced gland size means less sebum production.

FIG. 4 shows a line graph showing on the x axis time as time zero (“day 1’) and at one to six month periods, and on the y axis the average particle size D90 in microns for the Table 6 SLN particles formulation A, B and C and D, as determined using laser light scattering.

FIG. 5 shows photographs (top row taken five days after the formulations were prepared and bottom row taken two months after the formulations were prepared) of the Table 6 A, B, C and D formulations permitting visual inspection of these tazarotene-loaded SLN formulations over a two month period.

FIGS. 6A and 6B show two graphs presenting the results of differential scanning calorimetry (“DSC”) of tazarotene solid lipid (Compritol) solid lipid particles formulated as set forth in Table 10 (middle column).

FIGS. 7A and 7B show two graphs and an SEM photograph further characterizing the FIG. 6 tazarotene solid lipid (Compritol) particles with the Table 10 Compritol formulation (middle column).

FIGS. 8A and 8B show two graphs and two SEM photographs characterizing the tazarotene solid lipid (Crodamol) particles with the Table 10 Crodamol formulation (right hand side column).

FIG. 9 shows a flow chart summarizing a process for making 50 to 200 gram batches of the TazSLG of the present invention.

FIG. 10 shows two bar graphs the x axis of each being for in either the SLG2 formulation or in the SLG2-2 formulation, on the left hand side of the x axis, the particular tazarotene degradation (by oxidative reduction of the tazarotene) by product (an impurity therefore) “832”, and on the right hand side of each x axis total impurities, and on the y axis the weight % of such particular or total impurities in the indicated formulation. The left right hand side graph is for time zero, and the left hand side graph is as measured at time at plus one month.

FIG. 11 shows a schematic diagram of the equipment setup for the manufacture of TazSLNs within the scope of the invention.

FIG. 12 shows a particle size distribution graph showing on the x axis particle size (diameter) and on the y axis the % of the volume of the particles made with a particular particle size.

FIG. 13 shows a bar graph that presents the results of a one month dermal tolerability (i.e. skin irritation) study (in five groups of minipigs) of the ten tazarotene containing formulations and five vehicle formulations (the vehicles containing no tazarotene), shown on the x axis. Each pig in each of the five groups received topical administration to a different dermal area of a vehicle, that vehicle as part of the indicated tazarotene containing formulation, and the commercially available Tazorac gel. The y axis represents the observed percentage of erythema frequency.

FIGS. 14A, 14B, 14C, and 14D show three bar graphs and a line graph showing scores of erythema (graph A), eschar (graph B) and edema (graph C) in each minipig used for study after the one month application of the shown SLG2 related formulations. In graph D plasma tazarotene concentration as measured after dosing on day 28 is presented.

### DESCRIPTION

The invention is based on the discovery of stable compositions which when topically applied to the dermis (especially to the face) can be used to effectively treat certain dermal diseases and conditions, such as acne, with reduced side effects.

Unless specific definitions are provided, the nomenclatures utilized in connection with, and the laboratory procedures and techniques of analytical chemistry, synthetic organic and inorganic chemistry described herein are those known in the art. Standard chemical symbols are used interchangeably with the full names represented by such symbols. Thus, for example, the terms “hydrogen” and “H” are understood to have identical meaning. Standard techniques may be used for chemical syntheses, chemical analyses, and formulation.

The invention provides topical dermal compositions including a plurality of nanoparticles, wherein the particles include a biodegradable lipid, and a retinoid, or a pharmaceutically acceptable salt, ester, or amide thereof; wherein the particles have an average diameter between about 0.1 μm and about 10 μm.

In some embodiments, the particles have an average diameter no greater than about 5 μm. In some embodiments, the particles have an average diameter no greater than about 4 μm. In some embodiments, the particles have an average diameter no greater than about 1 μm.

Unless stated otherwise in this application, esters are derived from the saturated aliphatic alcohols or acids of ten or fewer carbon atoms or the cyclic or saturated aliphatic cyclic alcohols and acids of 5 to 10 carbon atoms. Examples include aliphatic esters derived from lower alkyl acids and alcohols, and phenyl or lower alkyl phenyl esters.

The term “amide” has the meaning classically accorded that term in organic chemistry. In this instance it includes the unsubstituted amides and all aliphatic and aromatic mono- and di-substituted amides. Examples include the mono- and di-substituted amides derived from the saturated aliphatic radicals of ten or fewer carbon atoms or the cyclic or saturated aliphatic-cyclic radicals of 5 to 10 carbon atoms. In one embodiment, the amides are derived from substituted and unsubstituted lower alkyl amines. In another embodiment, the amides are mono- and disubstituted amides derived from the substituted and unsubstituted phenyl or lower alkylphenyl amines. One may also use unsubstituted amides.

“Acetals” and “ketals” include the radicals of the formula-CK where K is (—OR)₂. Here, R is lower alkyl. Also, K may be —OR₇O— where R₇ is lower alkyl of 2-5 carbon atoms, straight chain or branched.

The term “alkyl,” by itself or as part of another substituent, means, unless otherwise stated, a straight (i.e. unbranched) or branched carbon chain, or combination thereof, which may be fully saturated (referred to herein as a “saturated alkyl”), mono- or polyunsaturated and can include di- and multivalent radicals, having the number of carbon atoms designated (e.g. “C₁-C₁₀” means one to ten carbons). Typical alkyl groups include, for example, methyl, ethyl, propyl, isopropyl, butyl, isobutyl, tertiary butyl, pentyl, hexyl and the like. The term “lower alkyl” refers to a C1-C6 alkyl group (e.g. methy, ethyl, propyl, isopropyl, n-butyl, sec-butyl, tert-butyl, pentyl, hexyl, and others identifiable to a skilled person). An “alkoxy” is an alkyl attached to the remainder of the molecule via an oxygen linker (—O—).

The term “aryl” means, unless otherwise stated, an aromatic substituent of 3 to 14 atoms (e.g. 6 to 10) which can be a single ring or multiple rings (e.g., from 1 to 3 rings) which may be fused together (i.e. a fused ring aryl) or linked covalently. A fused ring aryl refers to multiple rings fused together wherein at least one of the fused rings is an aryl ring (e.g., phenyl, 1-naphthyl, 2-naphthyl, or 4-biphenyl). The term “heteroaryl” refers to aryl groups (or rings) that contain one or more (e.g., 4) heteroatoms selected from N, O, and S, wherein the nitrogen and sulfur atoms are optionally oxidized, and the nitrogen atom(s) are optionally quaternized, the remaining ring atoms being carbon. The heteroaryl may be a monovalent monocyclic, bicyclic, or tricyclic (e.g., monocyclic or bicyclic) aromatic radical of 5 to 14 (e.g., 5 to 10) ring atoms where one or more, (e.g., one, two, or three or four) ring atoms are heteroatom selected from N, O, or S.

The terms “cycloalkyl” and “heterocycloalkyl,” by themselves or in combination with other terms, represent, unless otherwise stated, non-aromatic cyclic versions of “alkyl” and “heteroalkyl”, respectively (e.g., having 4 to 8 ring atoms). Additionally, for heterocycloalkyl, a heteroatom can occupy the position at which the heterocycle is attached to the remainder of the molecule. Pharmaceutically acceptable salts of a retinoid are also contemplated for use in the practice of the invention. A pharmaceutically acceptable salt is any salt which retains the activity of the parent compound and does not impart any deleterious or untoward effect on the subject to which it is administered and in the context in which it is administered.

Pharmaceutically acceptable acid addition salts of a retinoid are those formed from acids which form non-toxic addition salts containing pharmaceutically acceptable anions, such as the hydrochloride, hydrobromide, hydroiodide, sulfate, or bisulfate, phosphate or acid phosphate, acetate, maleate, fumarate, oxalate, lactate, tartrate, citrate, gluconate, saccharate and p-toluene sulphonate salts.

Pharmaceutically acceptable salts can be derived from organic or inorganic bases. The salt may be a mono or polyvalent ion. Of particular interest are the inorganic ions, sodium, potassium, calcium, and magnesium. Organic salts may be made with amines, particularly ammonium salts such as mono-, di- and trialkyl amines or ethanol amines. Salts may also be formed with caffeine, tromethamine and similar molecules. Where there is a nitrogen sufficiently basic as to be capable of forming acid addition salts, such may be formed with any inorganic or organic acids or alkylating agent such as methyl iodide. Preferred salts are those formed with inorganic acids such as hydrochloric acid, sulfuric acid or phosphoric acid. Any of a number of simple organic acids such as mono-, di- or tri-acid may also be used.

The nanoparticles included in the compositions of the invention have an average diameter no less than about 0.1 μm and no greater than about 10 μm

As used here, the term “about,” when used in connection with a value, means that the value may not differ by more than 10%. Hence, “about 10 μm” includes all values within the range of 9 μm to 11 μm.

In one embodiment, the nanoparticles of the invention have a maximum average diameter of about 10 μm. In another embodiment, the nanoparticles of the invention have a maximum average diameter of about 9 μm. In another embodiment, the nanoparticles of the invention have a maximum average diameter of about 8 μm. In another embodiment, the nanoparticles of the invention have a maximum average diameter of about 7 μm. In another embodiment, the nanoparticles of the invention have a maximum average diameter of about 6 μm. In another embodiment, the nanoparticles of the invention have a maximum average diameter of about 5 μm. In another embodiment, the nanoparticles of the invention have a maximum average diameter of about 4 μm. In another embodiment, the nanoparticles of the invention have a maximum average diameter of about 3 μm. In another embodiment, the nanoparticles of the invention have a maximum average diameter of about 2 μm. In another embodiment, the nanoparticles of the invention have a maximum average diameter of about 1 μm.

In another embodiment, the nanoparticles of the invention have a maximum average diameter less than about 1 μm. In another embodiment, the nanoparticles of the invention have a maximum average diameter of about 0.9 μm. In another embodiment, the nanoparticles of the invention have a maximum average diameter of about 0.8 μm. In another embodiment, the nanoparticles of the invention have a maximum average diameter of about 0.7 μm. In another embodiment, the nanoparticles of the invention have a maximum average diameter of about 0.6 μm. In another embodiment, the nanoparticles of the invention have a maximum average diameter of about 0.5 μm. In another embodiment, the nanoparticles of the invention have a maximum average diameter of about 0.4 μm. In another embodiment, the nanoparticles of the invention have a maximum average diameter of about 0.3 μm. In another embodiment, the nanoparticles of the invention have a maximum average diameter of about 0.2 μm. In another embodiment, the nanoparticles of the invention have a maximum average diameter of about 0.1 μm.

In one embodiment, the nanoparticle is shaped like a cylindrical rod. The inventors refer to such particles as “microcylinders,” even though they may have an average diameter in the nanometer range (that is, about 100 nm to about 999 nm). The microcylinders of the invention have a maximum average diameter and maximum average length such that no one such dimension is greater than about 10 μm. In other embodiments, the particles of the invention are of different geometry, such as fibers or circular discs; any geometry falls within the scope of the invention, as long as the average of any single dimension of the particle exceeds about 10 μm.

In one embodiment, the microcylinders of the invention have a maximum average diameter of about 10 μm. In another embodiment, the microcylinders of the invention have a maximum average diameter of about 9 μm. In another embodiment, the microcylinders of the invention have a maximum average diameter of about 8 μm. In another embodiment, the microcylinders of the invention have a maximum average diameter of about 7 μm. In another embodiment, the microcylinders of the invention have a maximum average diameter of about 6 μm. In another embodiment, the microcylinders of the invention have a maximum average diameter of about 5 μm. In another embodiment, the microcylinders of the invention have a maximum average diameter of about 4 μm. In another embodiment, the microcylinders of the invention have a maximum average diameter of about 3 μm. In another embodiment, the microcylinders of the invention have a maximum average diameter of about 2 μm. In another embodiment, the microcylinders of the invention have a maximum average diameter of about 1 μm.

In another embodiment, the microcylinders of the invention have a maximum average diameter less than about 1 μm. In another embodiment, the microcylinders of the invention have a maximum average diameter of about 0.9 μm. In another embodiment, the microcylinders of the invention have a maximum average diameter of about 0.8 μm. In another embodiment, the microcylinders of the invention have a maximum average diameter of about 0.7 μm. In another embodiment, the microcylinders of the invention have a maximum average diameter of about 0.6 μm. In another embodiment, the microcylinders of the invention have a maximum average diameter of about 0.5 μm. In another embodiment, the microcylinders of the invention have a maximum average diameter of about 0.4 μm. In another embodiment, the microcylinders of the invention have a maximum average diameter of about 0.3 μm. In another embodiment, the microcylinders of the invention have a maximum average diameter of about 0.2 μm. In another embodiment, the microcylinders of the invention have a maximum average diameter of about 0.1 μm.

In one embodiment, the microcylinders have a maximum average length of about 10 μm, about 9 μm, about 8 μm, about 7 μm, about 6 μm, about 5 μm, about 4 μm, about 3 μm, about 2 μm, about 1 μm, about 0.9 μm, about 0.8 μm, about 0.7 μm, about 0.6 μm, about 0.5 μm, about 0.4 μm, about 0.3 μm, or about 0.2 μm.

The size and geometry of the nanoparticles can also be used to control the rate of release, period of treatment, and drug (i.e. a retinoid) concentration. Larger particles will deliver a proportionately larger dose, but, depending on the surface to mass ratio, may have a slower release rate.

The retinoid of the invention can be in a particulate or powder form. In one embodiment, the retinoid itself consists of particles having the dimensions described above.

In another embodiment, a retinoid (such as tazarotene) is combined with a biodegradable lipid. In one embodiment, the retinoid is from about 1% to about 90% by weight of the composition. In another embodiment, the retinoid is from about 5% to about 85% by weight of the composition. In another embodiment, the retinoid is from about 10% to about 80% by weight of the composition. In another embodiment the retinoid is from about 15% to about 75% by weight of the composition. In one embodiment the retinoid comprises about 1%, about 5%, about 10%, about 15%, about 20%, about 25%, about 30%, about 35%, about 40%, about 45%, about 50%, about 55%, about 60%, about 65%, about 70%, about 75%, about 80%, about 85%, or about 90% of the composition.

Suitable materials (i.e. as the lipid constituent of the nanoparticles and as the gel constituent for the vehicle for the lipid nanoparticles) for use in the dermally applied compositions of the present invention include those materials which are biocompatible with the skin so as to cause no substantial irritation or other side effects. In one embodiment, such materials are at least partially biodegradable. In another embodiment, such materials are completely biodegradable.

Examples of useful materials include, without limitation, lipids such as Crodamol MM, Crodamol SS, myristyl myristate, myrisistyl laurate (Ceraphyl 424), triglycerides of C₁₀-C₁₈ and of C₁₀-C₂₂ fatty acids (for example Gelucire 43/01), and propylene glycol monopalmiteostearate (Monosteol). Other useful lipids include lipids derived from and/or including organic esters and organic ethers, which when degraded result in physiologically acceptable degradation products. Also, as a component of the gel vehicle, polymeric materials derived from and/or including, anhydrides, amides, orthoesters and the like, by themselves or in combination with other monomers, can be used. The polymeric materials may be addition or condensation polymers, advantageously condensation polymers. The polymers can be cross-linked or non-cross-linked, for example not more than lightly cross-linked, such as less than about 5%, or less than about 1% of the polymeric material being cross-linked. For the most part, besides carbon and hydrogen, the polymers will include at least one of oxygen and nitrogen, advantageously oxygen. The oxygen may be present as oxy, e.g. hydroxy or ether, carbonyl, e.g. non-oxo-carbonyl, such as carboxylic acid ester, and the like. The nitrogen may be present as amide, cyano and amino. The polymers set forth in Heller, CRC Critical Reviews in Therapeutic Drug Carrier Systems, Vol. 1, CRC Press, Boca Raton, Fla. 1987, pp 39-90 (Biodegradable Polymers in Controlled Drug Delivery), the contents of which are incorporated herein by reference, which describes encapsulation for controlled drug delivery, may find use in the present compositions.

Other additional polymers (as a component of the vehicle or carrier) include, for example, polymers of hydroxyaliphatic carboxylic acids, either homopolymers or copolymers, and polysaccharides, lipid nanoparticle, and mesoporous silica nanoparticle. Polyesters of interest include polymers of D-lactic acid, L-lactic acid, racemic lactic acid, glycolic acid, polycaprolactone, and combinations thereof. Generally, by employing the L-lactate or D-lactate, a slowly eroding polymer or polymeric material is achieved, while erosion is substantially enhanced with the lactate racemate.

Among the useful polysaccharides are, without limitation, calcium alginate, and functionalized celluloses, particularly carboxymethylcellulose esters characterized by being water insoluble, a molecular weight of about 5 kD to 500 kD, for example.

Other polymers of interest include, without limitation, polyesters, polyethers and combinations thereof which are biocompatible and may be biodegradable and/or bioerodible.

Some preferred characteristics of the polymers or materials for use in the present invention may include biocompatibility, compatibility with the therapeutic compound, ease of use of the polymer in making the compositions of the present invention, a half-life in the physiological environment of at least about 6 hours, preferably greater than about one day, and water insolubility.

The biodegradable lipids which form the nanoparticles are desirably subject to enzymatic or hydrolytic instability. The biodegradable lipid of the composition of the invention may comprise a mixture of two or more biodegradable lipids. For example, the composition may comprise a mixture of a first biodegradable lipid and a different second biodegradable lipid. One or more of the biodegradable lipids may have terminal acid groups.

Release of a drug (i.e. tazarotene) from an erodible lipid is the consequence of several mechanisms or combinations of mechanisms. Some of these mechanisms include desorption from the systems surface, dissolution, diffusion through porous channels of the hydrated polymer and erosion. Erosion can be bulk or surface or a combination of both.

One example of a composition of the invention includes tazarotene within a biodegradable lipid nanoparticle matrix. The composition system may have an amount of tazarotene from about 0.005 wt percent to about 1% or about 5% by weight of the system.

The release of tazarotene from the composition may include an initial burst of release followed by a gradual increase in the amount of tazarotene released, or the release may include an initial delay in release of tazarotene followed by an increase in release. When the biodegradable lipids substantially completely degraded, the percent of tazarotene that has been released is about one hundred percent.

It can be desirable to provide a relatively constant rate of release of tazarotene from the particles. However, the release rate may change to either increase or decrease depending on the formulation of the encapsulating nanoparticle. In addition, the release profile of tazarotene may include one or more linear portions and/or one or more non-linear portions. In one embodiment, the release rate is greater than zero once the system has begun to degrade or erode.

The lipid nanoparticles of the invention can be monolithic, that is, having the active agent or agents (i.e. a retinoid) homogenously distributed through the lipid matrix or encapsulated, where a reservoir of active agent is encapsulated by the lipid. The nanoparticle can be either monolithic with regard to retinoid distribution within the lipid or the retinoid can be encapsulated by the lipid. Greater drug release rate control can be afforded by the encapsulated, reservoir-type nanoparticles. Thus, nanoparticles can be prepared where the center may be of one material and the surface may have one or more layers of the same or a different material, where the layers may be cross-linked, or of a different molecular weight, different density or porosity, or the like.

The proportions of tazarotene and lipid in a nanoparticle can be empirically determined by formulating several drug delivery systems with varying proportions. A USP approved method for dissolution or release test can be used to measure the rate of release (USP 23; NF 18 (1995) pp. 1790-1798). For example, using the infinite sink method, a weighed sample of the nanoparticles is added to a measured volume of a solution containing 0.9% NaCl in water, where the solution volume will be such that the drug concentration is after release is less than 5% of saturation. The mixture is maintained at a temperature below the melting point of the lipid 37° C. and stirred slowly to maintain the nanoparticles in suspension. The appearance of the dissolved drug as a function of time may be followed by various methods known in the art, such as, for example, spectrophotometrically, HPLC, mass spectroscopy, and others identifiable to a skilled person, until the absorbance becomes constant or until greater than 90% of the drug has been released.

In addition to tazarotene and lipid, the nanoparticles and/or the vehicle disclosed herein can include effective amounts of buffering agents, preservatives and the like. Suitable water soluble buffering agents include, without limitation, alkali and alkaline earth carbonates, phosphates, bicarbonates, citrates, borates, acetates, succinates and the like, such as sodium phosphate, citrate, borate, acetate, bicarbonate, carbonate and the like. These agents advantageously present in amounts sufficient to maintain a pH of the system of between about 2 to about 9 and more, for example at about pH 4 to about 8. As such the buffering agent may be as much as about 5% by weight of the total drug delivery system. Suitable water soluble preservatives include sodium bisulfite, sodium bisulfate, sodium thiosulfate, ascorbate, benzalkonium chloride, chlorobutanol, thimerosal, phenylmercuric acetate, phenylmercuric borate, phenylmercuric nitrate, parabens, methylparaben, polyvinyl alcohol, benzyl alcohol, phenylethanol and the like and mixtures thereof. These agents can be present in amounts of from about 0.001% to about 5% by weight; in another embodiment, they can be present in amounts from about 0.01% to about 2% by weight.

In addition, the nanoparticles and or the vehicle can include a solubility enhancing compound provided in an amount effective to enhance the solubility of tazarotene relative to substantially identical systems without the solubility enhancing compound. For example, an implant can include a β-cyclodextrin, which is effective in enhancing the solubility of tazarotene. The β-cyclodextrin can be provided in an amount from about 0.5% (w/w) to about 25% (w/w) of the particle. In other embodiments, the β-cyclodextrin is provided in an amount from about 5% (w/w) to about 15% (w/w) of the particle.

Additionally, release modulators such as those described in U.S. Pat. No. 5,869,079, the contents of which are incorporated herein by reference, may be included in the nanoparticles. The amount of release modulator employed will be dependent on the desired release profile, the activity of the modulator, and on the release profile of the retinoid in the absence of modulator. Electrolytes such as sodium chloride and potassium chloride may also be included in the nanoparticles or in the vehicle. Where the buffering agent or enhancer is hydrophilic, it may also act as a release accelerator. Hydrophilic additives act to increase the release rates through faster dissolution of the material surrounding the drug particles, which increases the surface area of the drug exposed, thereby increasing the rate of drug bioerosion. Similarly, a hydrophobic buffering agent or enhancer dissolve more slowly, slowing the exposure of drug particles, and thereby slowing the rate of drug bioerosion.

Various techniques can be employed to produce the nanoparticles described herein. In one embodiment, particles are produced using a solvent evaporation process. Such a process can include steps of liquid sieving, freeze drying, and sterilizing the various composition compounds. In one embodiment, a retinoid and a lipid are combined with methylene chloride to form a first composition, and water and polyvinyl alcohol are combined to form a second composition. The first and second compositions are combined to form an emulsion. The emulsion is rinsed and/or centrifuged, and the resulting product dried. In a further embodiment, the emulsion undergoes an evaporation process to remove methylene chloride from the emulsion. For example, the emulsion can be evaporated for about 2 days or more. In this embodiment, the method includes sieving retinoid-containing nanoparticles in a liquid phase, as compared to a method which includes sieving retinoid-containing nanoparticles in a dry phase. This method can also comprise a step of freeze drying the sieved nanoparticles, and a step of packaging the freeze dried nanoparticles.

In one embodiment, the compositions of the invention can be used to treat conditions associated with excess sebum production. Such conditions include, for example, acne vulgaris, seborrhoeic dermatitis, and keratosis pilaris, as well as others identifiable to a skilled person. In another embodiment, the compositions of the invention can be used to treat those conditions in which it would be beneficial to suppress the function of the sebaceous gland. Such conditions include, for example, sebaceous cyst, sebaceous hyperplasia, sebaceous adenoma, and sebaceous gland carcinoma.

### EXAMPLES

The following examples illustrate embodiments of the invention and are not intended to limit the scope of the invention. Where units, amounts or concentrations are not set forth in the Tables infra the components or constituent in the Tables are shown as weight percent (wt. %).

### Example 1

### Making and Testing Tazarotene Containing SLNs

In this Example 1 set of experiments a number of tazarotene containing (as encompassed by or encapsulated by) SLN (SLN meaning solid lipid nanoparticle or solid lipid nanoparticles) were made and tested. It is known to topically apply tazarotene simply mixed into a gel or into a cream base or vehicle to treat acne and psoriasis, but so applying it to the skin can result in significant dermal irritation. It has been discovered by the Applicant that targeted delivery of a retinoid such as tazarotene, by preparing the tazarotene encompassed within biodegradable, solid at room temperature (20 to 25 degrees C., and in particular about 23 degrees C.) lipid nanoparticles, to the dermal sebaceous glands can enhance the efficacy of the treatment of a dermal condition such as acne by reducing sebum production by the glands and can also provide the added benefit of reducing irritation by decreasing exposure of the tazarotene per unit of time to the epidermis when the tazarotene is so topically applied to the skin formulated within SLN. Thus it has been determined that encapsulation of tazarotene in solid lipid nanoparticles can reduce exposure of tazarotene directly on the skin surface thus reducing the potential for skin irritation. Additionally, it has been discovered by the Applicant that the SLN-encapsulated tazarotene can improve the efficacy via the encapsulated system by preferentially depositing into the hair follicle. Once the encapsulated system is deposited into the hair follicle, the tazarotene is be released directly into the sebum producing sebaceous glands.

An important aspect of the invention is the creation of solid lipid nanoparticles (“SLN” or “particle” or “lipid particle”) for the delivery of tazarotene or other retinoid or other active pharmaceutical ingredient (e.g. an API such as bimatoprost) that can benefit from improved tolerability and efficacy by delivery into the hair follicle. Thus the Applicant determined how to make SLNs and used a hamster flank organ model to show sebaceous gland activity reduction.

### Formulation Compositions

As part of the development of SLN formulations comprising tazarotene several different lipids with varying melting points and chemical compositions were evaluated. The preferred lipid is a lipid into which can dissolve tazarotene, that can be easily dispersed in a water and surfactant system, and that can be processed through a high pressure fluid processor to thereby obtain SLN with a generally unimodal size distribution. Table 1 outlines the formulation compositions tested in this Example 1. Note that Table 1 identifies three suitable particular surfactants (polysorbate 80, Solutol HS 15 and Soluplus).

TABLE 1
Formulations comprising tazarotene as well the
tazarotene encapsulating solid lipids, and surfactants.

| | Formulation | | | | | | | |
|---|---|---|---|---|---|---|---|---|
| | 1 | 2 | 3 | 4 | 5 | 6 | 7 | 8 |
| | % | % | % | % | % | % | % | % |
| Ingredients | w/w | w/w | w/w | w/w | w/w | w/w | w/w | w/w |
| Tazarotene | 0.1 | 0.1 | 0.1 | 0.1 | 0.1 | 0.1 | 0.1 | 0.1 |
| Lipid Phase | | | | | | | | |
| excipients | | | | | | | | |
| Crodamol SS | 10 | 10 | 10 | — | — | — | — | — |
| Lipoid 90G | — | — | — | 5 | — | — | — | 5 |
| Crodamol MM | — | — | — | — | 10 | 10 | 10 | — |
| Surfactant | | | | | | | | |
| system | | | | | | | | |
| Polysorbate 80 | 2 | — | — | — | 2 | — | — | — |
| Solutol HS 15 | — | 1 | — | 1 | — | 1 | — | 1 |
| Soluplus | — | | 0.5 | — | — | — | 0.5 | — |
| Water | QS | QS | QS | QS | QS | | | |
| Particle Size | Bimodal | Unimodal | Separated | Separated | Unimodal | Unimodal | Unimodal | Separated |
| Distribution | | distribution | (8.1) | | distribution | distribution | distribution | |
| (numerical | | (0.31) | | | (0.13) | (0.32) | (16.1) | |
| value | | | | | | | | |
| represents the | | | | | | | | |
| D₉₀ in microns) | | | | | | | | |

TABLE 2
Effect of Surfactant Concentration on Tazarotene SLNs

| Formulation | 2 | 2a | 2b | 6 | 6a | 6b | 7 | 7a | 7b | 7c |
|---|---|---|---|---|---|---|---|---|---|---|
| Ingredients | % w/w | % w/w | % w/w | % w/w | % w/w | % w/w | % w/w | % w/w | % w/w | % w/w |
| Tazarotene | 0.1 | 0.1 | 0.1 | 0.1 | 0.1 | 0.1 | 0.1 | 0.1 | 0.1 | 0.1 |
| Crodamol SS | 10 | 10 | 10 | — | — | — | — | — | — | — |
| Crodamol MM | — | — | — | 10 | 10 | 10 | 10 | 10 | 10 | 10 |
| Solutol HS15 | 1.0 | 0.5 | 0.1 | 1.0 | 2.0 | 0.5 | — | — | — | — |
| Soluplus | — | — | — | — | — | — | 0.25 | 0.5 | 1.0 | 2.0 |
| Water | QS | QS | QS | QS | QS | QS | QS | QS | QS | QS |
| Particle Size | Unimodal | Unimodal | Separated | Unimodal | Unimodal | Unimodal | Separated | Separated | Separated | Separated |
| Distribution | distribution | distribution | | distribution | distribution | distribution | and then | and then | and then | and then |
| (numerical | (0.31) | (0.64) | | (0.32) | (0.25) | (0.96) | solidified | solidified | solidified | solidified |
| value | | | | | | | | | | |
| representst he | | | | | | | | | | |
| D₉₀ in microns) | | | | | | | | | | |

It was determined from the results set forth in Table 1 that two desirable (desirable because use of each of these two lipids resulted in desirable SLN diameters) lipids for making the SLN are firstly: myristyl myristate (available commercially as Crodamol MM). Myristyl myristate is also referred to as: tetradecanoic acid; tetradecyl ester; tetradecyl ester tetradecanoic acid; tetradecyl myristate; tetradecyl tetradecanoate; ceraphyl 424; Cyclochem MM; myristic acid, tetradecyl ester; tetradecyl myristate, and as; tetradecyl tetradecanoate, and is the ester of myristyl alcohol and myristic acid). The other desirable lipid is cetyl palmitate (C₃₂H₆₄O₂), which is available commercially as Crodamol SS.

Other lipids that are comparable to Crodamol MM and SS are also suitable as being expected to perform comparably can include myristyl laurate (Ceraphyl 424), triglycerides of saturated C10-C18 (or C10 to C22) fatty acids (such as Gelucire 43/01), isostearyl isostearate and propylene glycol monopalmiteostearate (Monosteol).

Table 2 summarizes the formulations and nanoparticle size information as influenced by the amount of surfactant added to the formulation. Thus nanoparticle size (measured as D90) decreased with increasing amount of surfactant added to the formulation.

Formulations from Table 1 and Table were selected 2 for an in vivo hamster flank organ model used to assess sebaceous gland reduction. The hamster study involves topical dosing of the selected formulations to the shaved flank skin with proper control on the right flank (the left flank of the same animal serves as the control). Treatment was conducted once a day for 5 days over a 4 week period. Following treatment the flank tissues were harvested, fixed, sectioned and sebaceous gland areas were measured with the treatment side being compared to control side by t-TEST. Drug treated groups were also compared with the vehicle treated group by one-way ANOVA. In more detail the protocol for the hamster flank organ model used was:
1. Male hamsters weighing about 110-120 grams each were used. They arrived at least 7 days before the study, were single-housed and randomized by weight.
2. The right side flank was shaved to expose the flank organ, removing as much hair as possible and wiped clean with Q-tip soaked with 70% ethanol.
3. The selected formulation was applied with a pipette and carefully spread over the flank organ. Each time before applying drug the flank organ area was wiped clean with Q-tip soaked with 70% ethanol.
4. Treatment continued 5 days/week for 26 days. The hair was shaved again if it grew back on the hamster flank organ.
5. The animals were sacrificed by CO₂, shave, clean and the flank organ was excised out.
6. The excised organ onto a paper card, placed into a thick cassett, overlain with a piece of sponge and closed. Air dry was carried for a few minutes before putting it into 10% formalin (buffered) for fixation.
7. The middle of the organ was cut to make 15-20 mm slices which were put onto glass slides and stained with Hematoxylin and Eosin.
8. The slides were scanned with NanoZoomer to obtain clear pictures and to measure the sebaceous gland areas with the NanoZoomer software.
9. The statistics analysis used was pair wise t-TEST to compare treated side vs. the untreated control side and one-way ANOVA analysis was used to compare drug treated group vs. vehicle treated group.

The formulations studied in the hamster model are listed in Table 3 below:

TABLE 3
Tazarotene formulations studied in hamster flank model.
Lipid to drug ratio based on formulation 6 in table 1.

| Formulation | 9 | 10 | 11 | 12 |
|---|---|---|---|---|
| Tazarotene | 0 | 0.01 | 0.03 | 0.06 |
| Crodamol MM | 10 | 1 | 3 | 6 |
| Solutol HS15 | 0.25 | 0.025 | 0.075 | 0.15 |
| Water | QS 100% (water phase comprised of PBS solution) | | | |
| Particle Size | 1.9 μm | 1.9 μm | 1.9 μm | 1.9 μm |
| (D90) | | | | |

TABLE 4
Tazarotene formulations studied in hamster flank model.
Lipid to drug ratio based on formulation 6 in Table 1.

| Formulation | 13 | 14 | 15 | 16 |
|---|---|---|---|---|
| Tazarotene | 0 | 0.01 | 0.03 | 0.005 |
| Crodamol MM | 10 | 1 | 3 | 0.5 |
| Solutol HS15 | 0.5 | 0.050 | 0.15 | 0.0025 |
| Water | QS 100% (water phase comprised of PBS solution) | | | |
| Particle Size | 0.4 μm | 0.4 μm | 0.4 μm | 0.4 μm |
| (D90) | | | | |

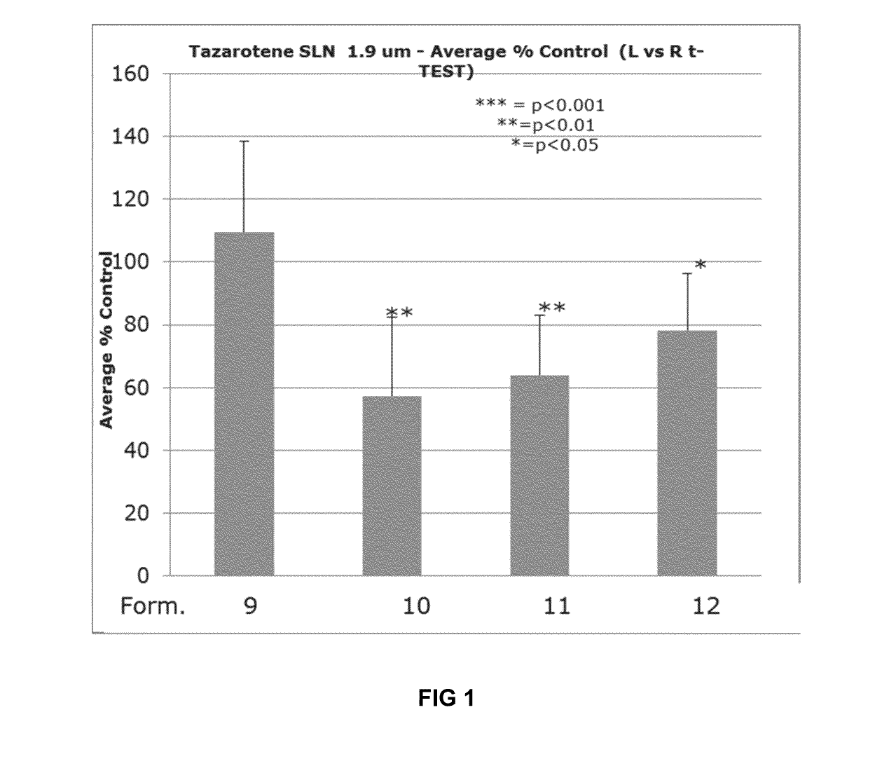

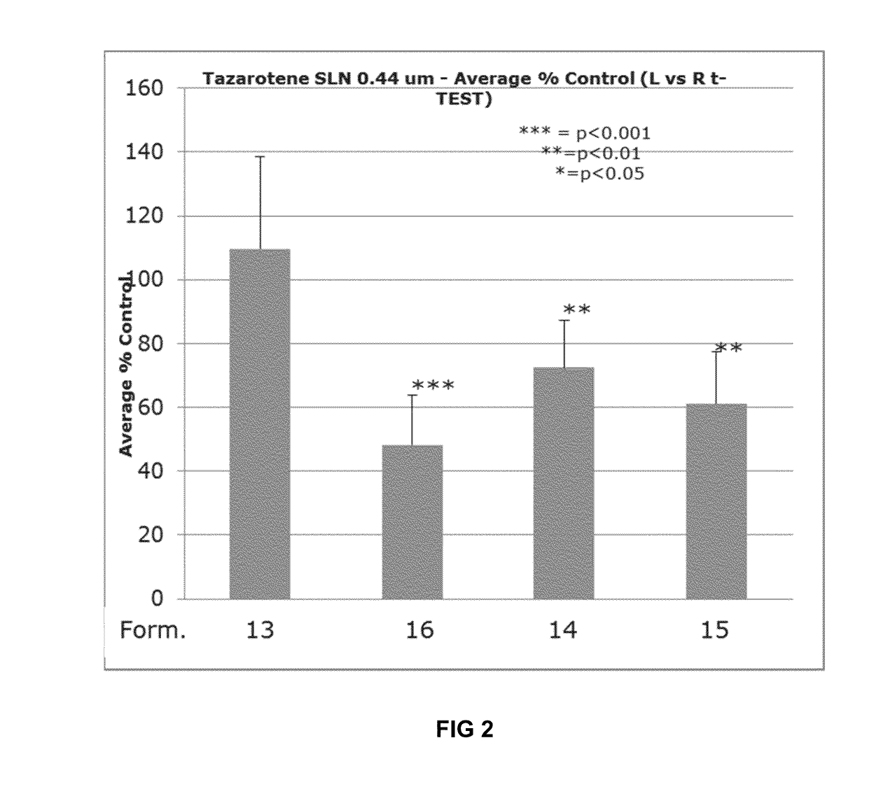

The results in FIGS. 1 and 2 show that the SLN formulations exhibited an effect that was independent of the size of SLN between the range studied. Further the concentration of tazarotene to elicit an effect is 0.005 wt % (formulation 16) which is 20 fold lower than the current marketed Tazorac formulations. Therefore the SLN formulations of the present invention with as little as 0.005 wt % tazarotene within the SLN can show a similar efficacy compared to the commercially available Tazorac formulation but with much lower skin irritation. The lower skin irritation is a result of a 20-fold reduction in tazarotene exposure on the skin surface and that the formulations were encapsulated within a lipid until deposited into the hair follicle and the drug is released into the sebum.

The SLN delivery system was further evaluated in the in vivo hamster flank organ model by studying the effects of accutane-loaded SLNs on sebaceous gland reduction. Accutane is also a retinoid like tazarotene that has been used (in a cream not in a SLN) for the treatment of acne and psoriasis. The formulations studied in the hamster model (based on the same protocol as previously described) are listed in Table 5 below.

TABLE 5
Accutane formulations studied in hamster flank model.
Lipid to drug ratio based on formulation 6 in table 2.

| Formulation | 17 | 18 | 19 |
|---|---|---|---|
| Accutane | 0.1 | 0.03 | 0.01 |
| Crodamol MM | 10 | 3.3 | 1 |
| Solutol HS15 | 0.25 | 0.083 | 0.025 |
| Water | QS 100% (water phase comprised of PBS solution) | | |
| Particle Size | 3.06 μm | 3.06 μm | 3.06 μm |
| (D90) | | | |

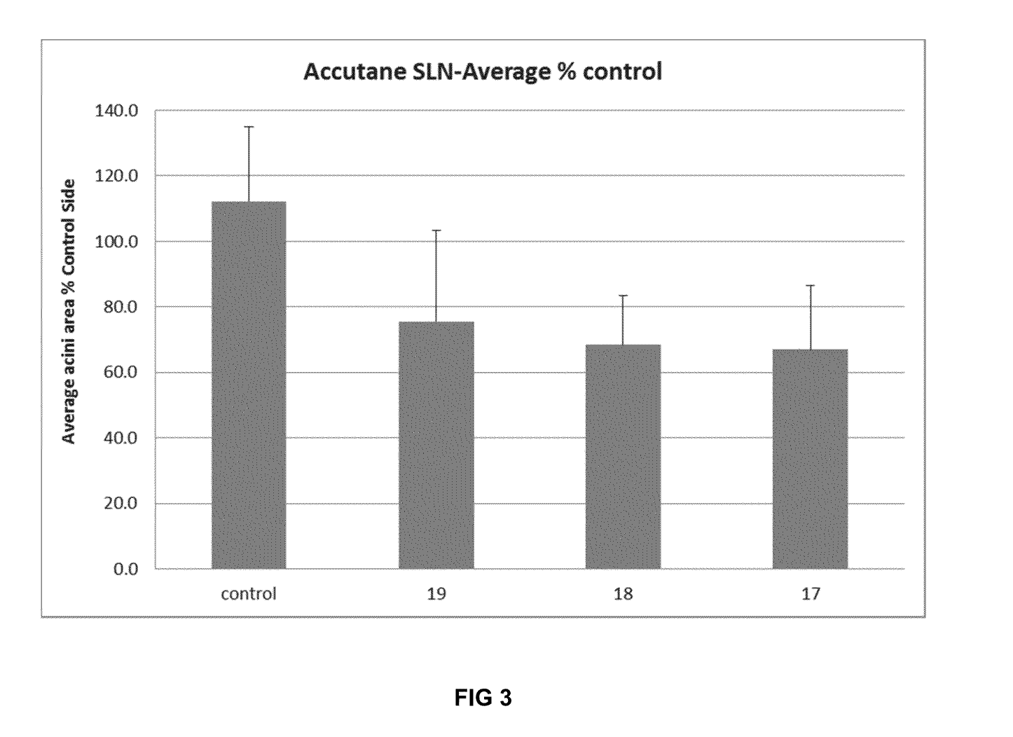

The results in FIG. 3 demonstrate that the accutane-loaded SLN formulations induced a significant reduction in sebaceous gland compared to empty PLGA (biodegradable polymer) microspheres. A slight increased efficacy was observed between 0.01% and 0.03% accutane-loaded SLNs, but the sebaceous gland reduction for 0.03% and 0.1% accutane-loaded SLNs was comparable. These results support the application of SLNs for therapeutic delivery of a retinoid.

### Additional Evaluation

The impact of Solutol HS15 concentration on the SLN-tazarotene formulation stability was also studied. The stability of the formulations were evaluated based on the particle size and lipid crystallinity, as measured by laser light scattering and differential scanning calorimetry, respectively, over a two month period (Day 1, 1 month and 2 month time points). The formulations listed in Table 6 were analyzed for both properties, whereas the formulations listed in Table 7 were only studied for the latter.

TABLE 6
Tazarotene formulations evaluated for
particle sizes and lipid crystallinity.

| Formulation | A | B | C | D |
|---|---|---|---|---|
| Tazarotene | 0.1 | 0.1 | 0.1 | 0.1 |
| Crodamol MM | 10 | 10 | 10 | 10 |
| Solutol HS15 | 0.25 | 0.5 | 1 | 2 |

| | |
|---|---|
| Water | QS 100% |

TABLE 7
Thickened tazarotene formulations evaluated for lipid crystallinity.

| Formulation | E | F | G | H |
|---|---|---|---|---|
| Carbopol ETD 2020 | 0.25 | 0.25 | 0.25 | 0.25 |
| Trolamine | 0.375 | 0.375 | 0.375 | 0.375 |
| SLN Formulation A | QS 100% | — | — | — |
| SLN Formulation B | — | QS 100% | — | — |
| SLN Formulation C | — | — | QS 100% | — |
| SLN Formulation D | — | — | — | QS 100% |

TABLE 8
Tazarotene-loaded SLN formulations evaluated for
lipid crystallinity over a six month period.

| | Initial | 1 Month | 2 Months | 3 Months | 4 Months | 5 Months | 6 Months |
|---|---|---|---|---|---|---|---|
| Formulation | ΔH (J/g) | | | | | | |
| A | No peaks | No peaks | 0.101 | 2.691 | 3.239 | 3.797 | 5.999 |
| | detected | detected | | | | | |
| B | No peaks | No peaks | 0.189 | 0.536 | 0.768 | 1.109 | 1.675 |
| | detected | detected | | | | | |
| C | No peaks | No peaks | 0.209 | 0.428 | 0.449 | 0.645 | 1.088 |
| | detected | detected | | | | | |
| D | No peaks | No peaks | No peaks | No peaks | 0.0972 | 0.313 | 0.449 |
| | detected | detected | detected | detected | | | |

TABLE 9
Thickened tazarotene-loaded SLN formulations evaluated
for lipid crystallinity over a six month period.

| SLN Taz | Initial | 1 Month | 2 Months | 3 Months | 4 Months | 5 Months | 6 Months |
|---|---|---|---|---|---|---|---|
| Gels | ΔH (J/g) | | | | | | |
| E | 14.30 | 15.50 | 21.30 | 19.83 | 18.75 | 19.89 | 16.54 |
| F | 2.73 | No peaks | 2.350 | 3.600 | 3.059 | 3.758 | 3.839 |
| | | detected | | | | | |
| G | No peaks | No peaks | 0.405 | 0.5933 | 0.544 | 0.556 | 0.648 |
| | detected | detected | | | | | |
| H | No peaks | No peaks | 0.082 | No peaks | 0.0939 | 0.0409 | 0.1104 |
| | detected | detected | | detected | | | |

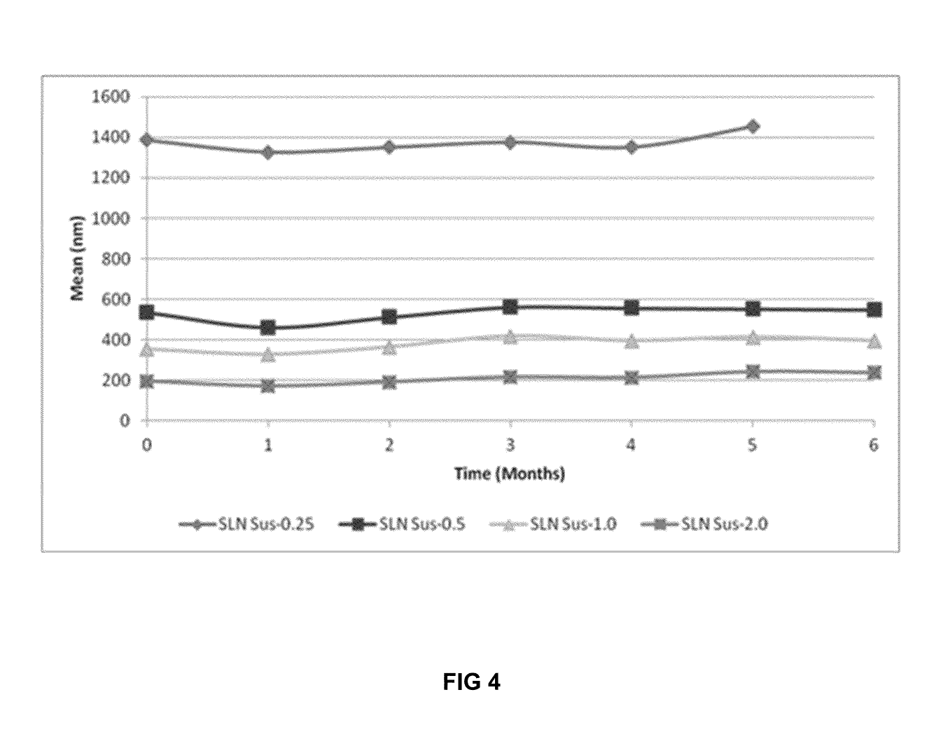

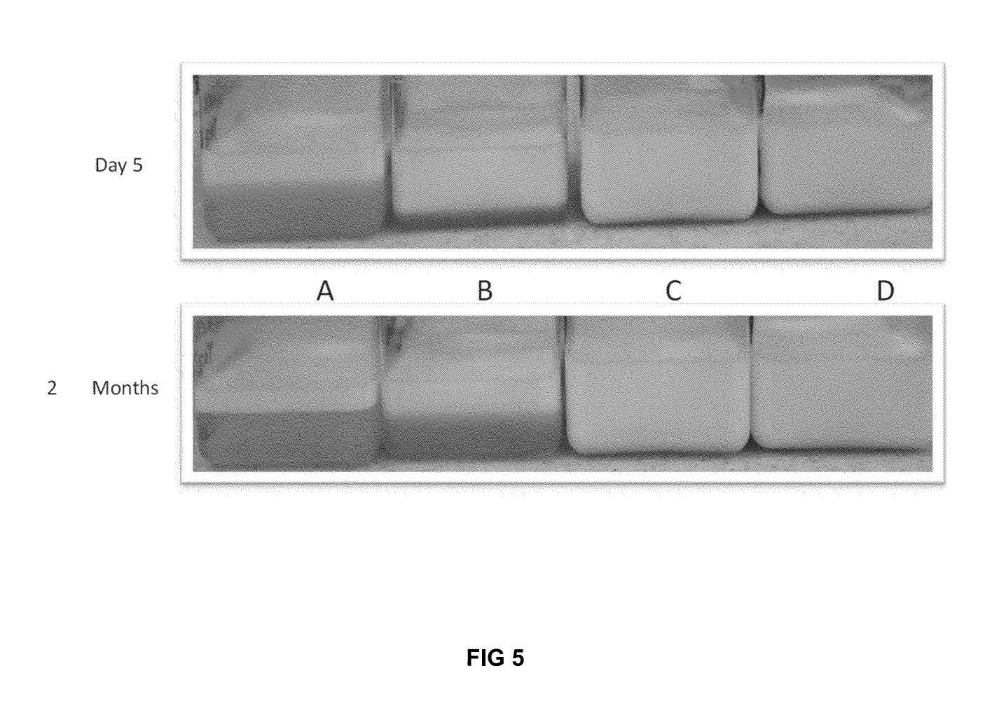

The stability of tazarotene-loaded SLN formulations were evaluated based on particle size, lipid crystallinity and visual inspection. First, increasing the concentration of Solutol HS15 corresponded with reduced SLN particle sizes. Second, the SLN formulations (Table 6) did not exhibit significant changes of particle size over a two month period (FIG. 4). On the other hand, lower amounts of Solutol HS15 (0.25% and 0.5%) in the SLN formulations resulted in phase separation, which was not observed for higher amounts (1% and 2%) up to six months. In addition, during the first month of storage at room temperature, the SLN formulations did not exhibit any lipid crystallinity over six months (Table 8). However, at two months, all the SLN formulations, except for the formulation D (with the highest amount of surfactant), began to show a slight amount of crystallized lipid at four months (Table 8). The amount of surfactant in the formulation also affected the lipid crystallinity of thickened SLNs. The addition of a thickener to the SLN formulations led to crystallized lipids for SLN formulations with lower amounts of surfactant (0.25% and 0.5%), whereas none were detected for the higher amounts (1% and 2%) (Table 9). Significantly, only after four months did all the thickened SLN formulations show some crystallized lipids, and the extent of lipid crystallinity was influenced by the surfactant amount present in the formulation (Table 9). These results demonstrate the importance of the present discovery of surfactant concentration upon SLN formulation stability.

### Example 2

### Tazarotene Containing SLN (Specific Lipid)

In this Example 2 it was determined that a preferred lipid for making the tazarotene incorporating SLN is glyceryl dibehenate or glyceryl behenate (available commercially as Compritol 888 ATO) because of the good compatibility of the lipid with the tazarotene, and for the desirable lipid melting point (72° C.) which ensures the solid state of the final particles when applied to the skin and into the skin hair follicles (32-37° C.) upon such targeted topical delivery of the SLN. The Applicant has previously studied site specific delivery of bimatoprost and tazarotene to the hair follicles, including particulate systems including micro/nano solid lipid particulates, PLGA microspheres (MS), mesoporous silica particulates, liposomes, neosomes, micelles, and nanoemulsions.

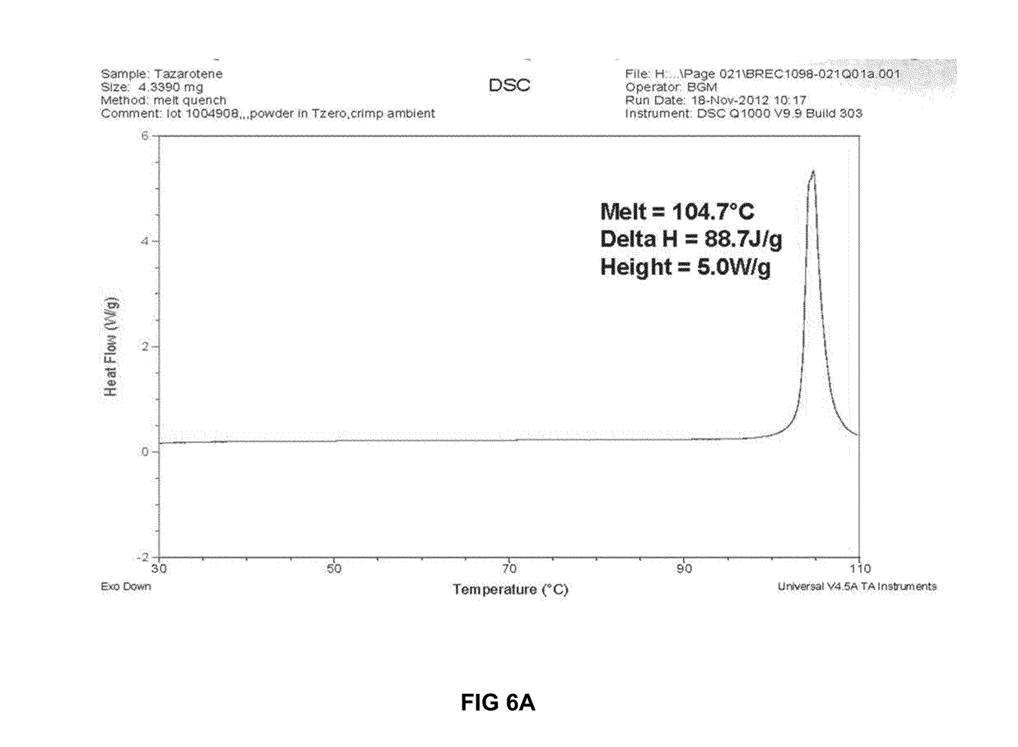

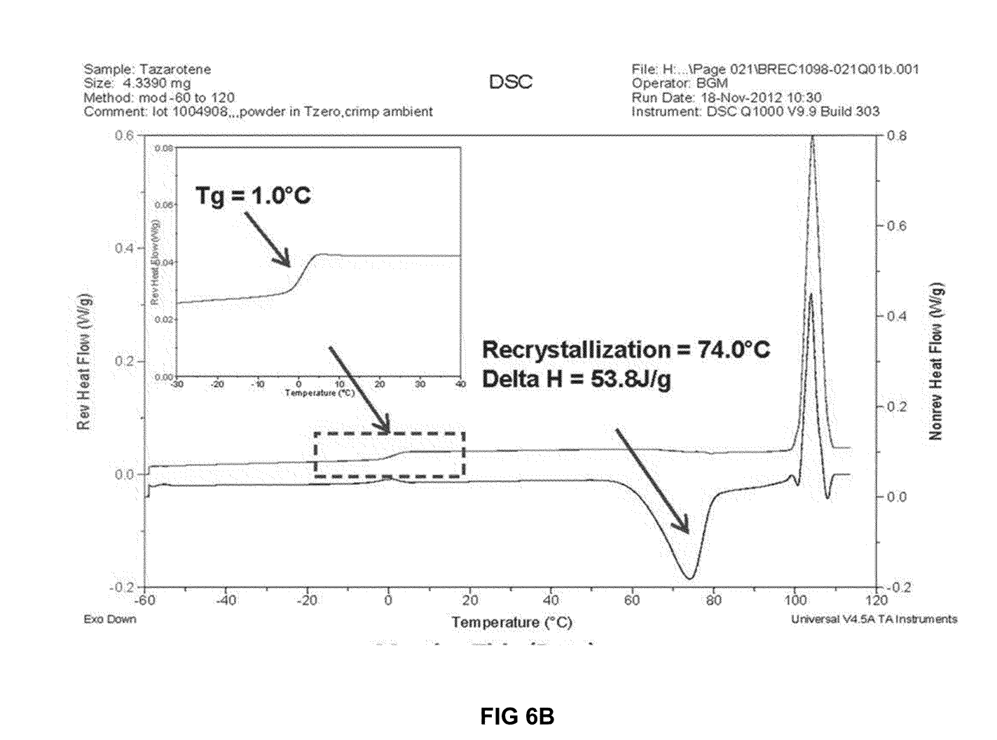

In this Example 2 tazarotene-containing solid lipid particulates comprising Compritol 888 ATO and/or Crodamol MM as the solid lipids used were developed and characterized. The formulations contained 5% of the solid lipid, 0.1% of Taz, and 0.5 Solutol HS-15, prepared using a homogenizing process. The encapsulation efficiency for both lipids was 100% or near 100% as shown in Table 10. The DSC for Compritol particulate system with is shown in FIG. 6.

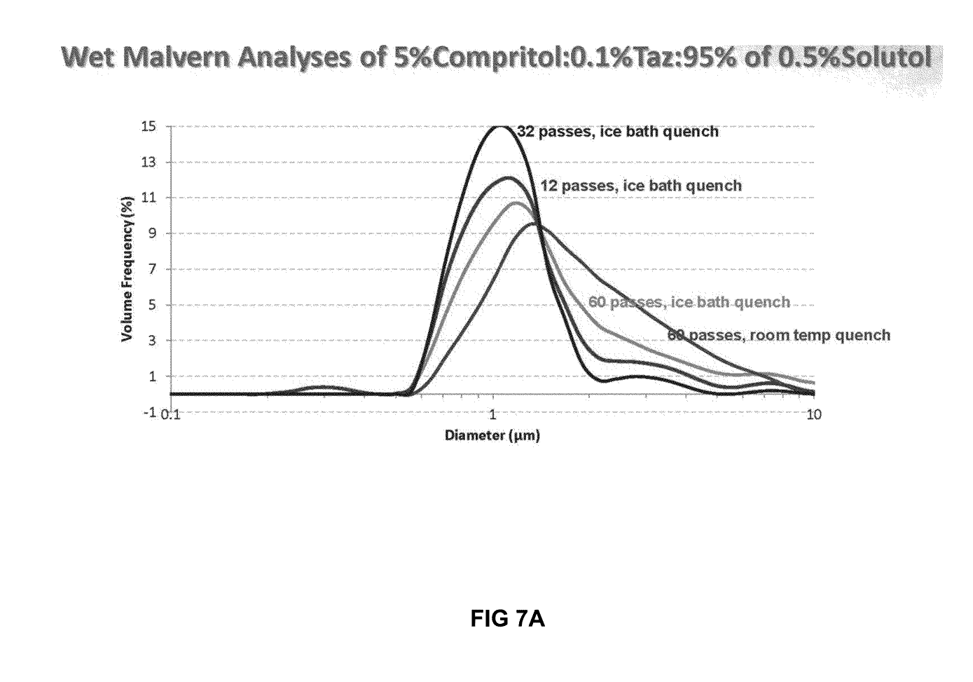

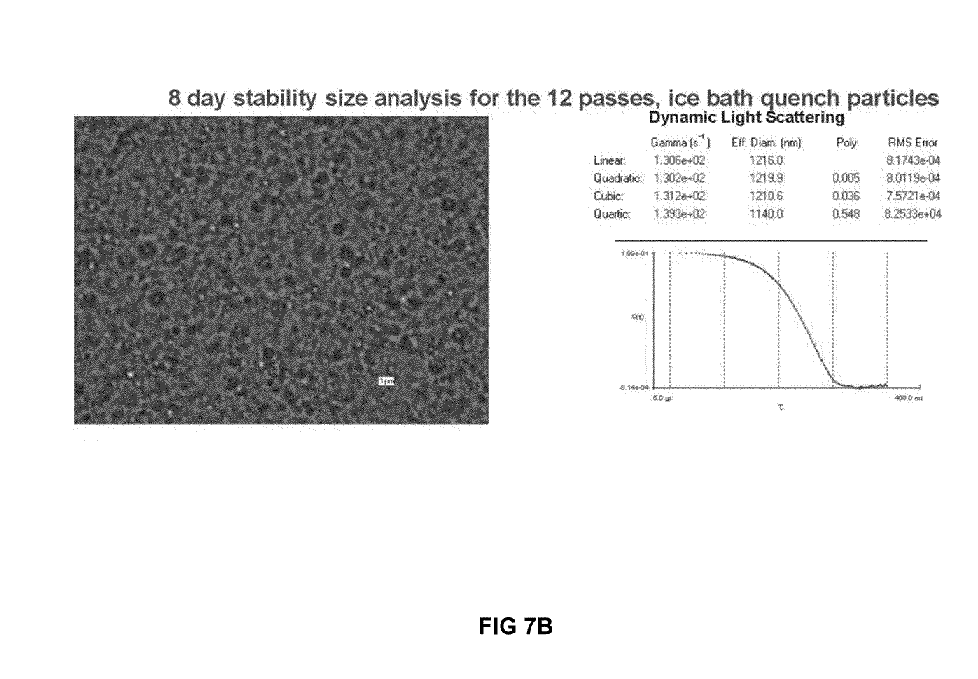

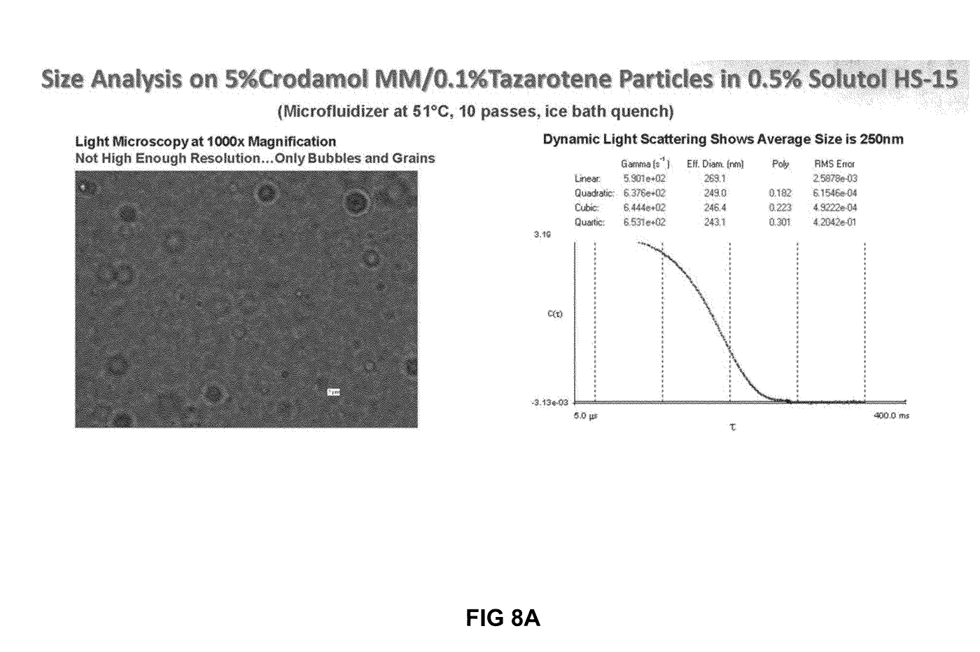

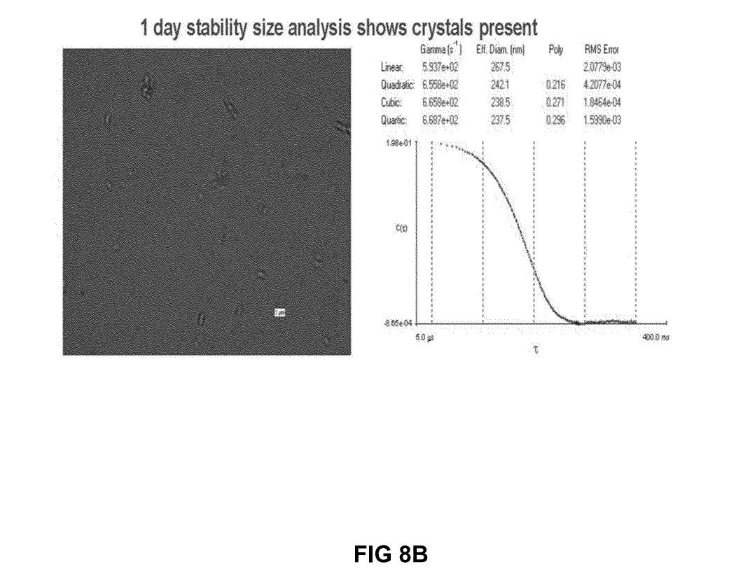

As shown in FIG. 7 and FIG. 8, the particle size distribution and the physical stability of the solid lipid system was substantially dependent on the lipids. Under the same composition and similar process conditions, the average particle size is about 250 nm for Crodamol system, and 1 μm for the Compritol system. There were drug crystals observed 24 hours after preparation of the Crodamol system while there were no crystals observed in the Compritol system for at least 2 weeks. In addition, the lipid with high melting point (Comprotol: about 72° C.) was likely to be more efficient for follicular drug delivery than the lipid melt at body temperature (Crodamol: about 36° C.). Therefore, the Applicant has discovered a more stable solid lipid particulate system loaded with tazarotene for storage, and more efficient for follicular drug delivery.

TABLE 10
Tazarotene encapsulation efficiency of
the solid lipid particulate systems

| | 5% Compritol:0.1% | 5% Crodamol:0.1% |
|---|---|---|
| | Taz:95% of 0.5% | Taz:95% of 0.5% |
| | Solutol Suspension | Solutol Suspension |
| Centrifugal | 0.24 μg | 0.00 μg |
| Filtrate | | |
| Wash Filtrate | 0.04 μg | 0.00 μg |
| Encapsulated Drug | 99.97% | 100.00% |
| (Based on | | |
| Theoretical | | |
| 100 μg in 1 mL | | |
| Sample) | | |

The procedures for encapsulating the tazarotene and determining potency are set forth below.

### Encapsulation Procedure

Pipetted 1 mL of suspension into tared Millipore Amicon Ultra 15 centrifugal devices, reweighed and spun at 4000×g for 1 hour at 22° C. Removed all of the filtrate and diluted it for HPLC analysis (1:1=150 μL+150 μL acetonitrile).
Pipetted 1 mL of Milli-Q H₂O into each of the centrifugal devices. Reweighed and spun again at 4000×g for 60 minutes at 22° C. Removed all of the filtrate and dilute it for HPLC analysis 1:1=150 μL+150 μL acetonitrile).
Rinsed out remaining solids from devices with five 1 mL volumes of Milli-Q H2O, using polyethylene transfer pipets. After each 1 mL rinse, the rinses were transferred to a 40 mL glass vial.
Added 20 mL tetrahydrofuran to the vial of remains, tightly capped (Teflon-lined cap) and vortexed on high for I minute. Also rinsed the transfer pipet with vial contents.
Diluted 1:5 in acetonitrile (150 μL+600 μL acetonitrile) into HPLC vials. The acetonitrile precipitated the Compritol from the THF, so spun it in microcentrifuge at 13,000 RPM for 1 minute at 22° C., then put supernatant into HPLC vial.

### Potency Procedure

Pipetted 1 ml of suspension into tared 40 ml glass vial. Reweighed vial to get exact sample weight.
Added 20 mL tetrahydrofuran to the vial of remains, tightly capped (Teflon-lined cap) and vortexed on high for I minute.
Dilute 1:5 in acetonitrile (150 μL+600 μL acetonitrile). The acetonitrile precipitated the Compritol from the THF, so spun it in microcentrifuge at 13,000 RPM for 1 minute at
22° C., then put supernatant into HPLC vial.

### Example 3

### Development and Testing of Tazarotene SLN and SLG Formulations

This Example 3 summaries a series of in vitro and in vivo experiments carried out which led to the Applicant's discovery and development of particular desirable formulations, including formulations comprising tazarotene encompassed by solid lipid nanoparticles and in a gel vehicle for effective treatment of dermal conditions. Thus, formulations comprising: tazarotene encapsulated within polymeric (i.e. PLGA) microspheres in a gel vehicle (“MSG”); formulations comprising tazarotene encapsulated within polymeric microspheres in a cream vehicle (“MSC”), and; formulations comprising tazarotene encapsulated or encompassed by solid lipid nanoparticles in a gel vehicle (referred to as “TazSLG” or simply as “SLG”) were made and evaluated. The weight % (% w/w) of the tazarotene in these various formulations was 0.1% or 0.04% tazarotene (“taz”). It was determined that the TazSLG formulations were the most desirable formulations. It should be understood that TazSLG comprises tazarotene encapsulated within solid lipid nanoparticles (TazSLN), and that the TazSLN are mixed in and dispersed throughout a non-lipid vehicle or carrier. Preferably the vehicle is a gel, such as a polymeric gel and/or a gel made using a polymeric gelling agent. Solid means a solid at room temperature.

Desirable TazSLG formulations can have for example 0.04 wt % and 0.1 wt % concentrations. The tazarotene containing TazSLG preferred formulations were chemically stable for more than 3 months at 25° C., and passed the preservative effectiveness test according to USP, EP-A and EP-B. They had no change in appearance. In non-clinical studies, the desirable TazSLG was the most effective formulation in reducing the size of sebaceous glands and the local irritation in the hamster model, and the most effective in improving dermal tolerance in the minipig model among MSG, MSC and other SLG (equivalently TazSLG) formulations. It is believed that the present TazSLG formulation is the first topical drug product under pharmaceutical development using tazarotene encapsulated solid lipid nanoparticulate technology. By these Example 3 experiments it was discovered two desirable formulations (with either 0.1% or 0.04% tazarotene % w/w concentration in the formulation), as shown in Table 11. The function of each component in the formulation is also described in Table 11, which shows that the preferred lipid used in the solid lipid phase was Compritol 888 ATO and that the preferred gel or gelling agent was Carbopol 974P.

TABLE 11
Two Desirable Formulations of TazSLG

| | | Regulatory | Placebo | 0.1% Taz | 0.04% Taz |
|---|---|---|---|---|---|
| Ingredients | Function | status | % w/w | % w/w | % w/w |
| Tazarotene | API | | 0 | 0.10 | 0.04 |
| Compritol* 888 ATO | Solid lipid phase | NF, EP | 7.0 | 7.0 | 7.0 |
| (Glyceryl behenate) | | | | | |
| Sodium thiosulfate | Antioxidant | USP/EP/JP | 0.10 | 0.10 | 0.10 |
| Butylated Hydroxytoluent (BHT) | Antioxidant | USP/EP | 0.05 | 0.05 | 0.05 |
| Butylated Hydroxyanisole (BHA) | Antioxidant | USP/EP/JP | 0.05 | 0.05 | 0.05 |
| EDTA | Chelating agent | USP/EP/JP | 0.05 | 0.05 | 0.05 |
| Carbopol 974P | Thickener | USP/EP/JP | 0.35 | 0.35 | 0.35 |
| Solutol HS 15* | Surfactant | NF/USP/EP | 2.0 | 2.0 | 2.0 |
| Methylparaben | Preservative | USP/EP/JP | 0.20 | 0.20 | 0.20 |
| Propylparaben | Preservative | USP/EP/JP | 0.10 | 0.10 | 0.10 |
| Phenoxyethanol | Preservative | USP/EP | 1.0 | 1.0 | 1.0 |
| Tromethamine** | Neutralizer | USP/EP | ~0.40 | ~0.40 | ~0.40 |
| Purified Water | Solvent | USP/EP/JP | q.s. | q.s. | q.s. |

*Meets Polyoxyl 15 hydoxystearate USP, and Macrogol 15 Hydroxystearate EP
**pH 5.5-7.5

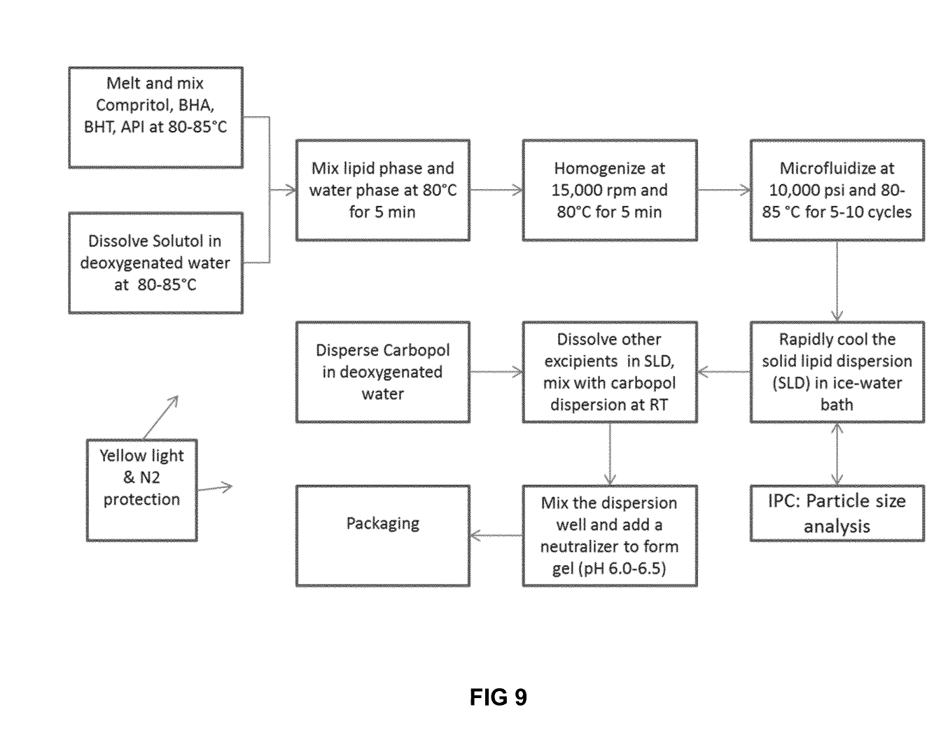

A unique manufacturing process for making the TazSLG formulations was also developed, as outlined in FIG. 9.

Detailed aspects of the manufacturing procedures outlined in FIG. 9 are as follows:

a. Preparation of TazSLN

A suspension of tazarotene encapsulating solid lipid nanoparticles (TazSLN) was made by a hot melt homogenization method using a microfluidizer M-100P (Microfluidics, MA) as follows:

1) A lipid phase comprising compritol ATO 888 (8.4%), as well as BHA (0.06%), BHT (0.06%) and the tazarotene (0.12%) was weighed and heated to 80-85° C., while a separate aqueous phase was made by mixing Solutol HS 15 (2.4%) and deoxygenated water which was then heated to the same temperature.
2) After agitation, the aqueous phase was poured into the lipid phase and mixed for about 5 minutes using a high speed magnetic stirring to form a milky mixture.
3) The mixture was then homogenized at 15,000 rpm for 5 minutes with a probe homogenizer (SilentCrusfier M, Heidolph), and then added into the microfluidizer, which was pre-warmed at 80-85° C. for 5 minutes.
4) The homogenized mixture was quenched to 5° C. temperature using a circulating water bath, thereby preparing an aqueous suspension of TazSLN, which was then collected into an amber glass bottle.
b. Preparation of Gel Phase

A 4% (by weight/weight) carbopol stock solution (containing carbopol and water only) was prepared by slowly and uniformly adding carbopol to the water phase to make a final carbopol concentration of 4.0% under slow agitation (stirring) to avoid the formation of lumps. Increase the agitation (stirring) to medium speed (at or greater than 500 rpm) afterwards to facilitate mixing. Keep agitation (stirring) for or for more than 3 hours until the formation of a uniform dispersion, thereby preparing the gel phase.

c. Preparation of TazSLG
1) The TazSLN suspension prepared (83.3%, 83.3 g for 100 g final gel), and added 0.2% methylparaben (0.2 g) and 0.1% propylparaben (0.1 g) was weighed into the TazSLN suspension and stirred using an overhead mixer (e.g. Heodoolph RZR 2051) at 300 rpm. EDTA Sodium (0.05 g, 0.05%) and sodium thiosulfate (0.1 g, 0.1%) were then added into the TazSLN suspension under stirring. Stirring was continued at room temperature for 1 hour or more until dissolved (by observing the bottom of the beaker for the added solids)
2) A 4% stock carbopol stock solution was then weighed to make the carbopol final concentration in the formulation of 0.35%. Phenoxylethanol (1.0%) was then weighed into the TazSLN suspension of step c. 1) above and stirred for about an hour until the formation of a uniform dispersion.
3) Drop by drop tromethanmine solution (500 mg/mL) was added into the mixed dispersion under stirring. The change of pH value was monitored using a recently calibrated pH meter. Then the final pH was adjusted to 5.5-6.0. Additional water was to make up to 100 g of the total weight of the formulation.
4) The mixture was stirred for another 60 minutes, thereby forming TazSLG.
5) The TazSLG was placed into a package, sealed and labeled.

In step a. 1) above Solutol HS 15 is Macrogol 15 hydroxystearate (Ph. Eur.) which in its USP monograph is known as Polyoxyl 15 hydroxystearate U. Solutol HS 15 can be used as a nonionic solubilizer as an emulsifying agent or as a surfactant and is made by reacting 15 moles of ethylene oxide with 1 cmole of 12-hydroxy stearic acid.

TABLE 12
Compositions of Five Desireable Tazarotene Containing Formulations

| | | % w/w | | | | |
|---|---|---|---|---|---|---|
| | | | MSG2 | | | |
| | | MSG1 | (Gel, 30% Gly, 30% | MSC | SLG1 | SLG2 |
| Ingredient | Function | (Gel, 60% Gly) | PEG400) | (Cream) | (LMP SL) | (HMP SL) |
| Tazarotene | API | 0.10 | 0.10 | 0.10 | 0.10 | 0.10 |
| R208 MS | Delivery system | 0.35 | 0.35 | 0.35 | | |
| Crodamol MM | Delivery system | | | | 10 | |
| Compritol 888 | Delivery system | | | | | 7.0 |
| Sodium thiosulfate | Antioxidant | 0.10 | 0.10 | 0.10 | 0.10 | 0.10 |
| EDTA | Chelating agent | 0.05 | 0.05 | 0.05 | 0.05 | 0.05 |
| Carbopo 974P | Thickerner | 0.35 | 0.35 | 0.4 | 0.35 | 0.35 |
| Carbomer 2020 | Emulsifier | | | 0.2 | | |
| Sorbitan monooleate | Emulsifier | | | 0.2 | | |
| Polysorbate 80 | Wetting agent | 0.2 | 0.2 | 0.2 | | |
| Solutol HS 15 | Surfactant | | | | | 2.0 |
| Poloxamer 407 | Wetting agent | 0.20 | 0.20 | | 2.0 | |
| Methylparaben | Preservative | 0.20 | 0.20 | 0.20 | 0.20 | 0.20 |
| Propylparaben | Preservative | 0.05 | 0.1 | 0.1 | 0.10 | 0.10 |
| Glycerin | Co-solvent | 60 | 30 | 10 | | |
| PEG 400 | Co-solvent | | 30 | 10 | | |
| Mineral oil | Oil phase | | | 8 | | |
| Cyclomethicone | Oil phase | | | 2 | | |
| Tromethamine | Neutralizer | pH 5.5-6.5 | | | | |
| Purified water | Solvent | Q.S. | Q.S. | Q.S. | Q.S. | Q.S. |

It was determined that a microparticle or nanoparticle system can be used for follicular targeted drug with improved efficacy and with reduced skin irritation resulting. The present invention is the first microparticle or nanoparticle system formulated for and effective for follicular drug (i.e. tazarotene) delivery technology. Thus the Applicant has developed tazarotene loaded microsphere dispersions and, tazarotene loaded solid lipid nanoparticles (SLN) in a gel (TazSLG) for reducing both size and activity of mammalian sebaceous glands, as demonstrated for example in a hamster flank organ model, was also developed. It was discovered that the effect of these particulate systems were superior to the effects of the commercially available TAZORAC gel, which does not contain any microparticles or nanoparticles, but only tazarotene dispersed in a gel.

Considering the complexity of microparticle and nanoparticulate systems and the potential interaction of microsphere systems with topical vehicles, gel dispersions were also studied. Thus, five micro/nano particulate formulations containing 0.1% tazarotene were made and evaluated, these five being the MSG1, MSG2, MSC, SLG1 and SLG2 formulations shown in Table 12. Using the commercially available 0.1% Tazorac® Gel formulation as the control (that is as compared to the results obtained in the same model system using the 0.1% Tazorac® Gel) the MSG1 and MSG2 formulations showed substantial reduction of skin irritation and some improvement in efficacy. MSC showed good efficacy but little improvement in skin irritation. Formulation SLG2 (equivalently TazSLG2) showed the best overall performance. To improve the stability of the SLG2 formulation to the SLG2 formulation there was added of 0.05% of butylated hydroxyanisole (BHA) and 0.05% of butylated hydroxytoluene (BHT), as shown in Table 11 (BHT is incorrectly spelled as butylated hydroxytoluent in Table 11). Deoxygenated water, yellow light and nitrogen protection were also applied in manufacture process to reduce the degradation of the tazarotene during the manufacturing process. This formulation (i.e. the Table 12 SLG2 formulation improved as noted in Table 11) is the desirable formulation SLG2-2. As a result of the reformulation and process changes for SLG2-2, chemical stability was considerably improved, and the impurity content was less than 0.9%. In addition, 1.0% phenoxyethanol was also incorporated in SLG2-2 to improve antimicrobial activity. As a result of formulation modification and process improvement, SLG2-2 passed 3 month stability and antimicrobial preservative effectiveness test (APET) against EP-A study. Thus two desirable formulations of the present invention of a retinoid (e.g. tazarotene) in solid lipid nanoparticles in a gel vehicle are shown in Table 11.

TABLE 13
Physicochemical Properties of the Preferred Retinoid Tazarotene

| | |
|---|---|
| Name: | Tazarotene |
| Chemical Name: | Ethyl 6-[(4,4-dimethylthiochroman-6-yl)ethynyl]nicotinate |
| Emp. Formula: | C₂₁H₂₃NO₂S |
| Molec. Wt.: | 351.46 |
| Indications: | psoriasis and acne vulgaris |
| Structure: | 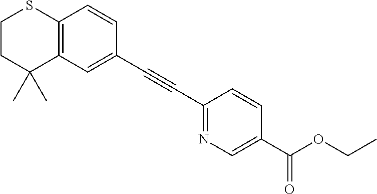 [SMILES: CCOC(=O)c1ccc(C#Cc2ccc3c(c2)C(C)(C)CCS3)nc1] |
| Melting Pt.: | 103-106° C. |
| pKa: | 1.5⁽¹⁾ |
| Distrib. Coef.: | log p = 4.3⁽²⁾ |
| Solubility in water: | <1 μg/mL |
| Solubility in isopropyl myristate: | 7.4 mg/mL |

Tazarotene is a member of the acetylenic class of retinoids, and is a retinoid prodrug which is converted to its active form, the cognate carboxylic acid of tazarotene, by rapid deesterification in animals, including in man. Tazarotenic acid binds to all three members of the retinoic acid receptor (RAR) family: RARα, RARβ, and RARγ but shows relative selectivity for RARβ, and RARγ and may modify gene expression. Common side effects include worsening of acne, increased sensitivity to sunlight, dry skin, itchiness, redness and in some cases extreme drying and cracking of skin. Solubility of tazarotene in Compritol ATO 888 (82° C.) was determined to be 3-4 weight, and no crystalline drug was detected by DSC or PXRD analysis (at 1% or at 4% in Compritol).

A desirable target average particle (population) size (diameter) for the tazarotene encapsulating solid lipid nanoparticles was about 1 micron to about 10 microns and in particular from about 2 microns to about 7 microns. The lipid used to make the solid lipid nanoparticles can be for example one of the Table 14 lipids.

TABLE 14
Suitable Lipids

| Common/Trade | | | Compendial |
|---|---|---|---|
| Name | Formal Name | MP (° C.) | Status |
| Beeswax, Super | Beeswax, White | 63 | USP/NF |
| Refined (SR) | | | |
| Cetyl alcohol | Cetyl alcohol | 45-52 | USP/NF |
| Capmul MCM | Medium chain | 37 | USP/NF |
| | mono/diglycerides | | |
| Cithrol | Glyceryl monostearate | 58 | USP/NF |
| Compitrol 888 ATO | Glycerol behenate | 72 | USP/NF |
| Crodamol MM | Myristyl myristate | 38 | JP |
| Crodamol SS | Cetyl esters | 44 | USP/NF |
| Phospholipon 90G | Phosphatidyl choline | None | Non- |
| | | | Compendial |
| Precirol ATO 5 | Glycer | 56 | USP/NF |
| | palmitoestearateyl | | |

Suitable surfactants present in the gel vehicle can be one of more of the surfactants shown in Table 15.

TABLE 15
Suitable Surfactants

| Common/Trade | | Compendial |
|---|---|---|
| Name | Formal Name | Status |
| Phospholipon 90G* | Phosphatidyl choline | Non-Compendial/ |
| | | GRAS |
| Polysorbate 80 | Polyoxyethylene sorbitan | USP/NF |
| | monooleate | |
| Poloxamer 188 | Polyethylene/polypropylene | USP/NF |
| | oxide copolymer | |
| Solutol HS15 | Polyoxyl 15 | USP/NF |
| | Hydroxystearate | |
| Soluplus | Polyvinyl caprolactam/ | Non-Compendial |
| | PVA/PEG copolymer | |

### TazSLG Development

The TazSLG formulations were formulated as set forth supra and tested in the hamster model. The TazSLN in gel (thereby forming TazSLG) dispersions were freshly prepared using a microfluidizer for the in vivo test. The average particle size was less than about 1 μm.

TABLE 16
TazSLG Components

Formulation

| Name | Placebo | 0.01 | 0.03 | 0.06 |
|---|---|---|---|---|
| Tazarotene | 0 | 0.01 | 0.03 | 0.06 |
| Crodamol MM | 6 | 1 | 3 | 6 |
| Solutol | 0.15 | 0.025 | 0.075 | 0.15 |

| | |
|---|---|
| Water* | QS 100% |

As noted above, the results from the hamster model showed that TazSLG reduced sebaceous glands, and also reduced skin irritation.

### TazSLG1 and TazSLG2

A desirable TazSLN comprised as the lipid myristyl myristate (i.e. Crodamol MM) and Poloxamer. It was also determined that because the solid lipid particles in the vehicle of the formulation have a relatively high melting point (greater than 37° C.) and therefore can assist retention of the integrity of the formulation after it's topical application to the skin (which facilitates penetration by the formulation into deep follicles), therefore a high melting point lipid such as glycerol behenate (i.e. Compitrol 888 ATO) can alternately be used in the SLG. Thus, using a combination of Compitrol and Solutol HS 15, SLN2 was formulated. Both SLN1 and SLN2 were physically stable for at least 2 weeks based on appearance and microscope images. The constituents of both the SLG1 and SLG2 formulations are shown by Table 17 (in the table “API” means active pharmaceutical ingredient).

TABLE 17
SLG1 and SLG2 Formulations

| | | SLG1 | SLG2 | |
|---|---|---|---|---|
| Ingredient | Function | % w/w | % w/w | Selection Criteria |
| Tazarotene | AP I | 0.10 | 0.10 | Used in the SLN at 0.1% or 0.04% |
| | | | | concentration |
| Crodamol MM | Delivery system | 10 | | Used in TazSLN1. Measured melting |
| | (vehicle) | | | point for this formulation was 32-36° C. |
| Compritol 888 | Delivery system | | 7.0 | Melting point in the vehicle was 73-76° C. |
| | | | | facilitates enhanced follicular delivery. |
| Sodium thiosulfate | Antioxidant | 0.1 | 0.1 | Used in Tazorac cream |
| EDTA | Chelating agent | 0.05 | 0.05 | Used in Tazorac products |
| BHT | Antioxidant | 0.05 | 0.05 | Used in Tazorac gel |
| BHA | Antioxidant | 0.05 | 0.05 | Used in Tazorac gel |
| Carbopo 974P | Thickener | 0.35 | 0.35 | Used in Tazorac gel |
| Polysorbate 80 | Wetting agent | | | |
| Solutol HS 15 | Surfactant | | 2.0 | Used in TazSLN2 formulation |
| Poloxamer 407 | Wetting agent | 2.0 | | Used in Tazorac gel |
| Methylparaben | Preservative | 0.20 | 0.20 | Replace benzyl alcohol (1%) used in |
| | | | | Tazorac products, due to the effect on |
| | | | | physical stability |
| Propylparaben | Preservative | 0.10 | 0.10 | Same as methylparaben |
| Phenoxyethanol | Preservative | 1.0 | 1.0 | Added for enhancing antimicrobial |
| | | | | activity |
| Tromethamine | Neutralizer | | | Used in Tazorac cream |
| Purified water | Solvent | | | |

### Modified Taz SLG

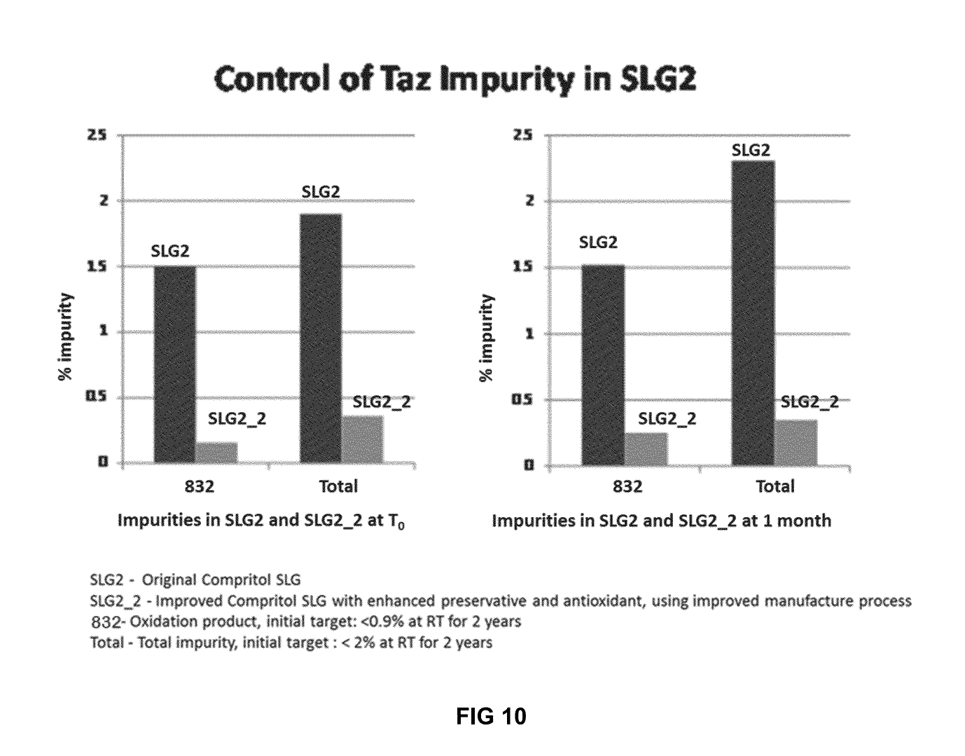

A first three month stability study using the initial TazSLG1 and tazSLG2 formulations resulted after the three months in the presence of undesirable oxidation products at levels greater than 1%. Therefore these initial formulation were modified (thus becoming the desirable TazSLG1-2 and TazSLG2-2 formulations) by addition of BHA and BHT to further prevent oxidation and by addition of phenoxyethanol to increase antimicrobial activity. Additionally the manufacture process was improved by using deoxygenated water, yellow light for reducing photo degradation and nitrogen protection to lower API degradation during manufacture. A second three month stability study of the two so modified TazSLG formulation, with the altered manufacturing process, shown significantly reduced degradation (as determined by the presence in the formulation of the amount of tazarotene oxidation product, as shown by FIG. 10) and less microbial activity, with the modified TazSLG2 being much more stable than the modified TazSLG1.

### Scale Up

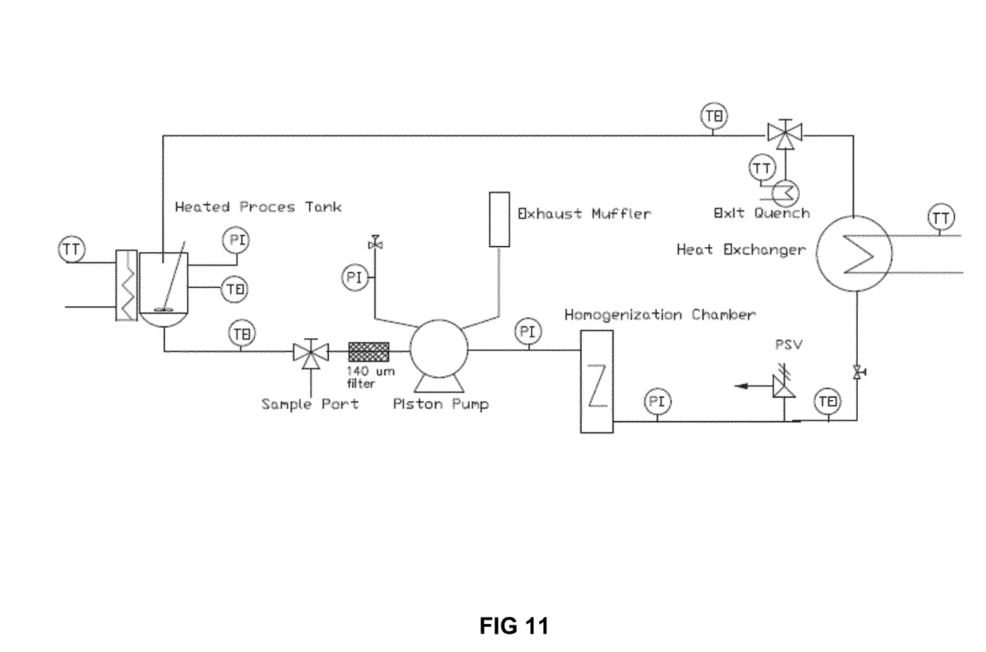

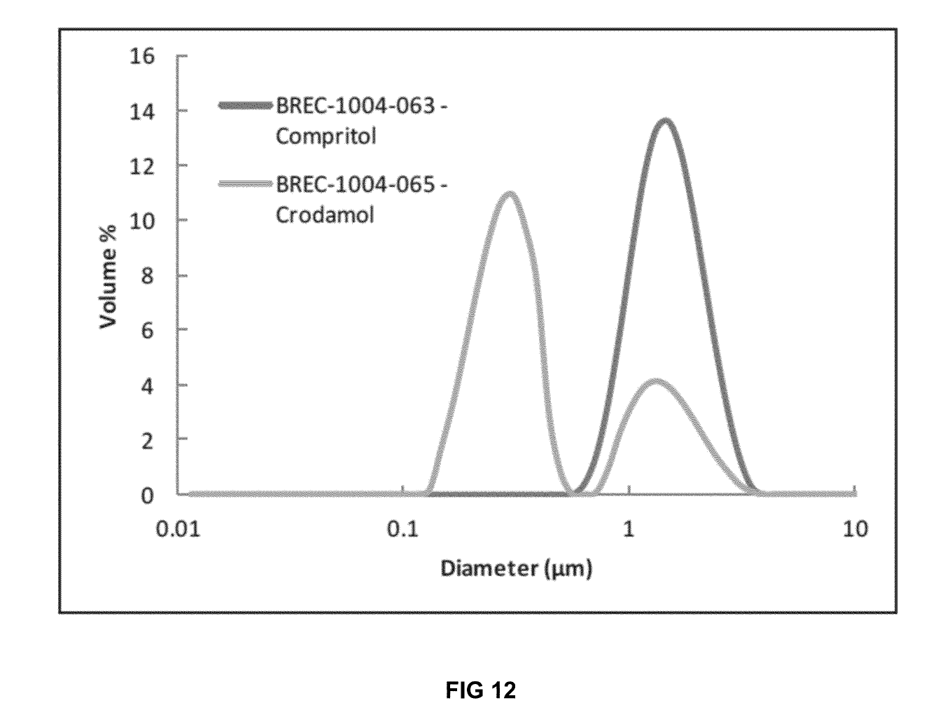

A scale up process for TazSLG was developed to permit increasing the amount of TazSLN made from 20 g to 1 to 2 kg batches. The particles size distribution of the dispersion and the manufacture process were shown in respectively FIGS. 12 & 11. In FIG. 11 “BREC-1004-063-Compritol” is the TazSLN2-2 formulation while “BREC-1004-065-Crodamol” is the TazSLN1-1 formulation.

### Further Development of TazSLG Formulations

The type and level of the excipients in the SLG formulations was further improved. For example, Compritol was selected primarily due to its high melting point. Other solid lipids with the melting point greater than 7° C. are also suitable for use in the TazSLG formulations. As noted supra three antioxidants were included in a desirable SLG formulation. Additionally, it was determined that SLN is best manufactured at 5-10° C. above the melting point of the lipid materials used. Therefore a SLN comprising Compritol ATO 888 was made at around 75-80° C. Furthermore, it was determined that use of 9500 psi during the TazSLN manufacturing produced the best formulations in terms of particle size distribution (i.e. when made at 9500 psi the particle size distribution of the SLNs was sharply centered at 1 micron. Further, it was determined that an optimal lipid content was 8.4 wt %. Thus it was possible to make up to 1-2 kg batches of the TazSLN1 and 2 formulations using a pressure of 9500 psi, tank temperature of 82° C., and number of passes 20.

Table 18 shows particle size distribution, viscosity and pH of six so made TazSLN2 formulations: SLG2 0.1%, GLP SLG2 0.1% (SLG2-2) and 0.04% (SLG2-9).

TABLE 18
Batch history of SLG2 manufactured

| | Batch | | | | Gel | |
|---|---|---|---|---|---|---|
| | Size | SLN size (μm) | | | viscosity | |
| Batch Description | grams | D10 | D50 | D90 | (Pa*s) | pH |
| Prototype SLG | 1000 | 1.1 | 2.0 | 4.1 | 39.0 | 5.6 |
| (0.1%) (Batch 1) | | | | | | |
| Prototype SLG | 250 | 0.4 | 1.0 | 3.9 | >200 | 5.4 |
| (0.1%) (Batch 2) | | | | | | |
| Prototype SLG | 1000 | 1.1 | 1.9 | 3.6 | NA | 5.6 |
| (0.04%) | | | | | | |
| GLP SLG (0.1%) | 250 | 1.0 | 4.1 | 8.0 | >200 | 5.6 |
| GLP SLG (0.04%) | 250 | 0.9 | 3.4 | 6.4 | >200 | 5.7 |
| Tergus SLN in | 300 | 0.6 | 1.1 | 2.0 | pending | pending |
| Boston *** | | | | | | |

***: 5 passes, 80° C., 10 kpsi, quenching, using M-110EH Microfluidizer

TABLE 19
Formulations of one SLN1 and nine SLN2 formulations

| | | % w/w | | | | | | | | | |
|---|---|---|---|---|---|---|---|---|---|---|---|
| Ingredient | Function | SLG1_2B | SLG2_2B | SLG2_3p | SLG2_3 | SLG2_4 | SLG2_5 | SLG2_6 | SLG2_7 | SLG2_8 | SLG2_9 |
| Tazarotene | API | 0.10 | 0.10 | 0 | 0.10 | 0.10 | 0.10 | 0.10 | 0.10 | 0.10 | 0.04 |
| Crodamol MM | Delivery system | 10 | | | | | | | | | |
| Compritol 888 | Delivery system | | 7.0 | 7.0 | 7.0 | 7.0 | 7.0 | 7.0 | 7.0 | 7.0 | 7.0 |
| EDTA | Chelating agent | 0.05 | 0.05 | 0.05 | 0.05 | 0.05 | 0.05 | 0.05 | 0.05 | 0.05 | 0.05 |
| Sodium thiosulfate | Antioxidant | 0.10 | 0.10 | 0.10 | 0.10 | 0.10 | 0.10 | 0.10 | 0.10 | 0.10 | 0.10 |
| BHT | Antioxidant | 0.05 | 0.05 | 0.05 | 0.05 | 0.05 | 0.05 | 0.05 | 0.05 | 0.05 | 0.05 |
| BHA | Antioxidant | 005 | 0.05 | 0.05 | 0.05 | 0.05 | 0.05 | 0.05 | 0.05 | 0.05 | 0.05 |
| Tocopherol | Antioxidant | | | | | | 0.01 | | | | |
| Ascorbic acid | Antioxidant | | | | | | | 0.10 | | | |
| Propyl gallate | Antioxidant | | | | | | | | 0.01 | | |
| Carbopol 974P | Thickener | 0.35 | 0.35 | 0.35 | 0.35 | 0.35 | 0.35 | 0.35 | 0.35 | 0.35 | 0.35 |
| Polysorbate 80 | Surfactant | | | | | 0.20 | | | | | |
| Solutol HS 1S | Surfactant | | 2.0 | 2.0 | 2.0 | 2.0 | 2.0 | 2.0 | 2.0 | 2.0 | 2.0 |
| Poloxamer 407 | Emulsifier | 2.0 | | | | | | | | | |
| Methylparaben | Preservative | 0.20 | 0.20 | 0.20 | 0.20 | 0.20 | 0.20 | 0.20 | 0.20 | 0.20 | 0.20 |
| Propylparaben | Preservative | 0.10 | 0.10 | 0.10 | 0.10 | 0.10 | 0.10 | 0.10 | 0.10 | 0.10 | 0.10 |
| Phenoxyethanol | Preservative | 1.0 | 1.0 | 1.0 | 1.0 | 1.0 | 1.0 | 1.0 | 1.0 | 0.5 | 1.0 |

| | | |
|---|---|---|
| Tromethamine | Neutralizer | pH 5.5-6.5 |
| Purified water | Solvent | q.s. |

In order to improve the stability of the TazSLG2 formulations two antioxidants BHA and BHT were added to the TazSLG formulations. Secondly, deoxygenated water and yellow light were employed to minimize the effect on API oxidization. This resulted in the SLG2-2 formulations after preparation and after one month of storage having less than 0.5% of oxidization product and total impurity. Additional anti-oxidants vitamin E (in formulation SLG2-5), vitamin C (in formulation SLG2-6) and propyl gallate (in formulation SLG2-7) were incorporated into the SLN formulations as shown in Table 19. These later 3 formulations were whitish gel after gel preparation. The “832” (an impurity resulting from oxidation of tazarotene) impurity level for SLG2-5 and SLG2-6 was only 0.2-0.3% at time zero (“T0”), and no other impurities were observed. The incorporation of propyl gallate resulted in that formulation of an 832 impurity of 0.26%, while the total impurity reached up to 0.92%. The 832 impurity level at month 2 was less than 0.9% for SLG2-5 and SLG2-6, irrespective of the storage temperature. The 832 impurity level in SLG2-7 formulation was also <0.9% at the end of 2 month at 25° C., but increased up to 1.1% at 40° C., which has exceeded the 0.9% limit. Finally, SLG2-2 was found to be the formulation with the best chemical stabilities.

To improve the dispersion of SLNs in the gel and prevent their aggregation 0.2% Tween 80 in tazSLG2-4 was used. In SLG2-8, 0.5% instead of 1.0% phenoxyethanol for antimicrobial activity were used. The 0.04% SLG2 showed improved efficacy and reduced irritation, thusly SLG2-9 was formulated as well. All the formulations, except the SLG2-2, had an oxidization level greater than 0.5% after 3 month storage. Although SLG2-2 had slightly higher oxidization level than SLG2-3 the 832 level was still below the 0.9% limit. Formulations stored at 40° C. led to higher levels of total impurities, but most of them, except SLG2-2 and SLG2-9, fell below 1.0%. SLG2-2 and SLG2-9 reached over 1.6% of total impurity, while their oxidization level reached 0.9% and 1.0%, respectively. Considering the drug strength, the absolute amount of the impurity in SLG2-9 should be 40% lower than that in SLG2-2. These data showed that the chemical stability of these formulations at 25° C. met the 3 month criteria.

### Physical Stability

The TazSLG formulations described herein both improved efficacy by reducing the sebaceous glands, and minimized the side effects like irritation by encapsulating drugs into micro/nano particles. Formulations were targeted for the hair follicles so that particles can enter the hair follies and provide a sustained release directly into the sebaceous gland. Thus the particle size and integrity of the particles are critical to the successful development of a product, and need to be assessed. The pH of all the formulations remained around 7.0 at both temperatures (25° C. and 40° C.), as measured by ASET. At 25° C., the viscosity of all the TazSLG2 related formulations increased, even reaching a 2-fold change at the end of 3 months. In contrast, tazSLG1 formulations had a decreased viscosity along with time. At 40° C., the viscosity of the SLG2 related formulations either remained plateaued (SLG2-2, SLG2-4, SLG2-8, SLG2-9) or increased only slightly (SLG2-3), while SLG1 formulations had a decreased viscosity at the end of 3 months. At 25° C., the solid form of Compritol solid lipid particles may slowly change and reach a new equilibrium, and the particles tended to form flocculates which increase the viscosity especially at low shear rate. The viscosity changed significantly in SLG2-2 but not in SLG2. The SLG1 comprising Crodamol showed decreased viscosity along with time, even at 40° C. storage conditions. The SLG1-2 formulation has a viscosity of 8 Pa·s, while SLG2-2 has a viscosity above 126 Pa·s. 10% crodamol was used in SLG1-2, while in SLG2-2, 7% compritol was used.

### Follicular Delivery in Pig Ear Model

TABLE 20
Pig Ear Hair Follicle Taz Formulation Penetration Data Summary

| | Positive | Avg of | Avg |
|---|---|---|---|
| | Follicles/ | Deepest | Distance of |
| | Total | Distance | Concentrated |
| Sample | Follicles | (μm) | Area (μm) |
| Taz SLN 0.4 μm | 15/15 | 353 | 195 |
| Taz SLN 1.9 μm | 12/12 | 288 | 132 |
| SLG2-2 | 37/49 | 445 | 284 |
| | (75.5%) | | |
| SLG2-4 | 18/18 | 389 | 234 |
| | (100%) | | |

Excellent delivery of the tazarotene from both the TazSLN and TazSLG formulations into the hair follicles of mammals was found. For example follicular delivery of the formulations ex vivo into pig ears was studied. The SLN comprising the lipid crodamol had an average particle size of 0.4 μm, while the SLN comprising the lipid compritol had an average particle size of 1.9 μm. It was discovered that both these TazSLNs formulations effectively penetrated into numerous and essentially all the hair follicles present in the skin of the pig ears they were applied to. The smaller SLN penetrated deeper into the pig ear follicles. The TazSLG-2 penetrated the hair follicles deeper than did the SLG2-4. The only difference between SLG2-2 and SLG2-4 is that SLG2-4 comprised 0.2% Tween. Thus, the experiments as summarized by Table 20 showed that the solid lipid nanoparticles (SLN) used as either TazSLN or as the SLN from the TazSLG formulations in either case the SLNs penetrated into the porcine hair follicles.

### Effect on Flank Sebaceous Gland and Skin Irritation in Hamster Model

An in vivo hamster (shaved) flank efficacy study was performed using MSG, MSC and TazSLG formulations was carried out. The formulation which comprised 0.5 wt % carbopol, 10 wt % glycerin, 15 wt % propylene glycol, and 10 wt % PEG 400, achieved the highest reduction of sebum production by follicular sebaceous gland (about a 40% reduction as compared to control). An alternate formulation which the same ingredients except for having 1.0 wt % carbopol caused about 27% sebum reduction of sebum production, as compared to control. This indicated the strong effect of viscosity, with lower viscosity being preferred to obtain a higher sebum reduction. The MSG F1 (PEG), formulation which is equivalent to the MSG1 formulation comprised 0.5% carbopol and 60% glycerin, and caused a sebum reduction of 29.2%. MSG2 contained 30% glycerin and 30% PEG 400 and 0.35% carbopol, but had a sebum reduction of only 12.4%. It is believe that the propylene glycol facilitates the penetration of MS into the hair follicles. Taz SLG1 and MSC formulations achieved better efficacy than Tazorac® Gel (˜30% reduction in sebum production versus 22%). For PLGA MS, the limited ability to achieve higher and more consistent drug release is an obstacle to better efficacy.

The effects of formulations to cause after topical application an increase in epidermal thickness and irritation scores was also examined. MSC, aged MSC, Tazorac® Gel, MSG2, F5LV formulations achieved similar epidermal thickness (about 60 μm), while MSG1 and F5HV yielded a thickness of about 50 μm. These results showed that the use of vehicle or co-solvents themselves may increase the epidermal thickness.

Additionally, five TazSLG formulations were tested in the hamster model, these being the SLG1 0.04%, SLG1 0.1%, SLG2 0.04%, SLG2 0.1% and Tazorac 0.1% commercial gel formulations. The SLG2 formulation showed very good efficacy at both the 0.04% and 0.1% tazarotene concentrations, and is a preferred formulation (causing about sebaceous inhibition of the sebum producing follicular glands). In contrast, SLG1 0.1% had an inhibition of 23.5%, while SLG1 0.04% 31.1%. Interestingly, all the SLG formulations, irrespective of the drug strength, showed similar epidermal thickness as caused by the Tazorac gel 0.04% Notably while the Tazorac gel had an average irritation score of 9.2, the SLG1 formulations showed a score less than 5 and for the lower (0.04 wt %) tazarotene concentration SLG2 there was even lower irritation. The SLG1 also worked better at the lower 0.04% concentration too.

TABLE 21
Inhibition of Sebum Production.

| | Formulation | | | | |
|---|---|---|---|---|---|
| | SLG1 | SLG1 | SLG2 | SLG2 | Tazorac |
| | 0.04% | 0.1% | 0.04% | 0.1% | 0.1% |
| % | 31.1 | 23.5 | 39.5 | 43.4 | 21.8 |
| sebaceous | | | | | |
| inhibition | | | | | |

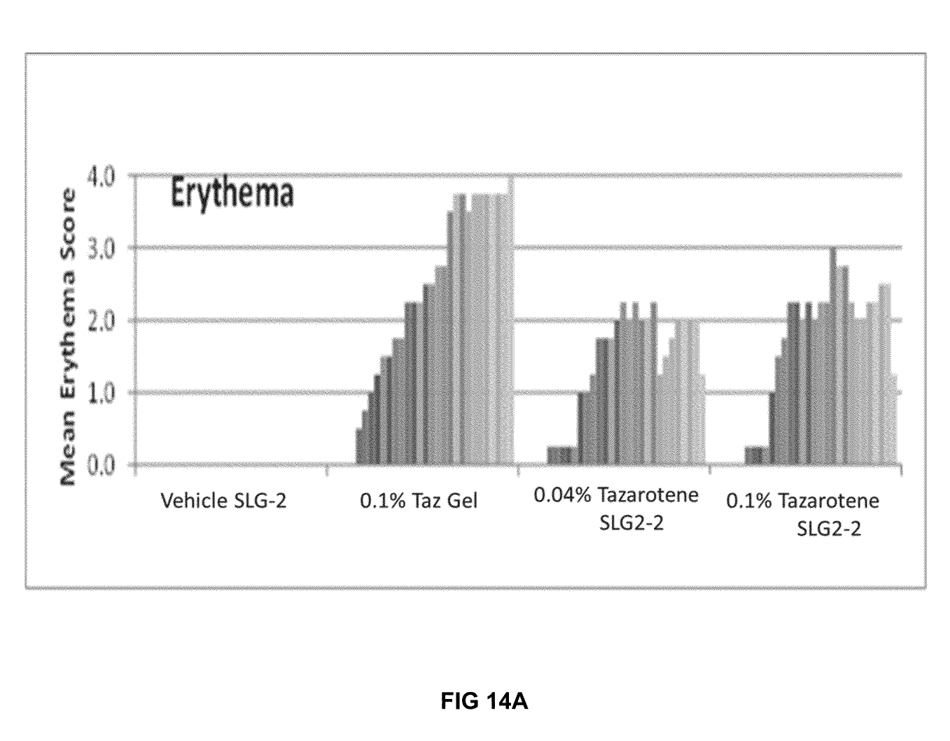

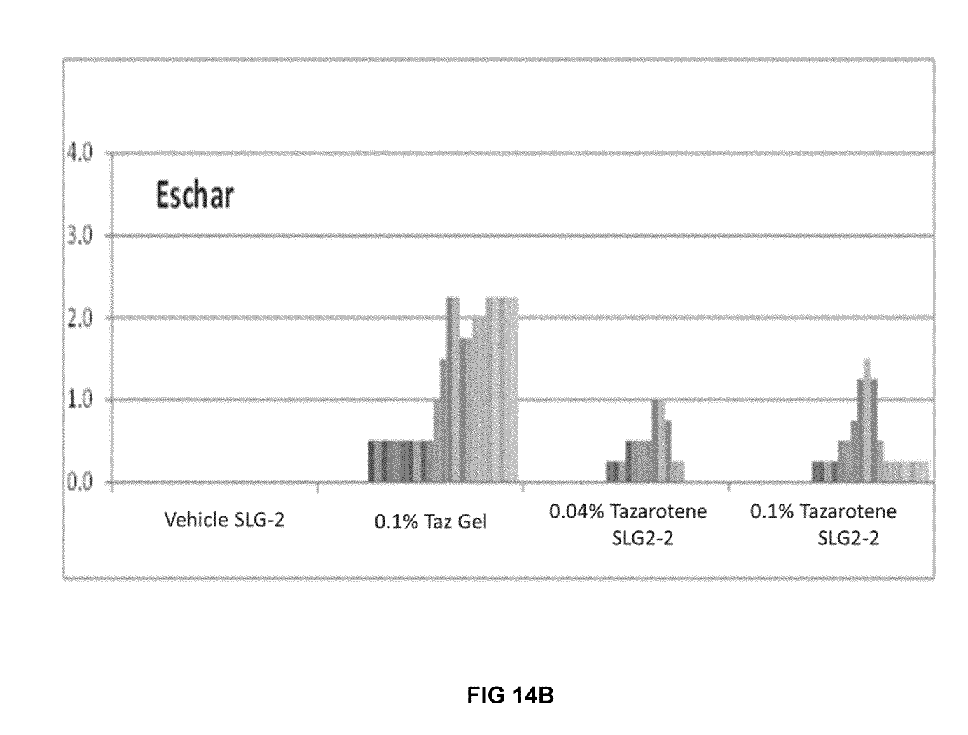

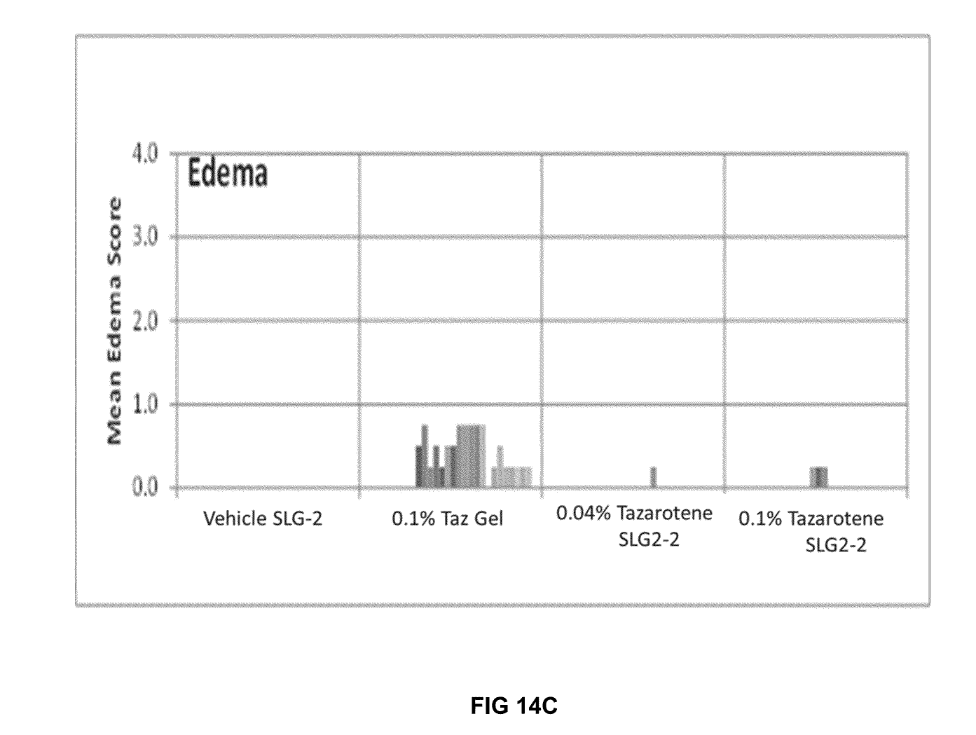

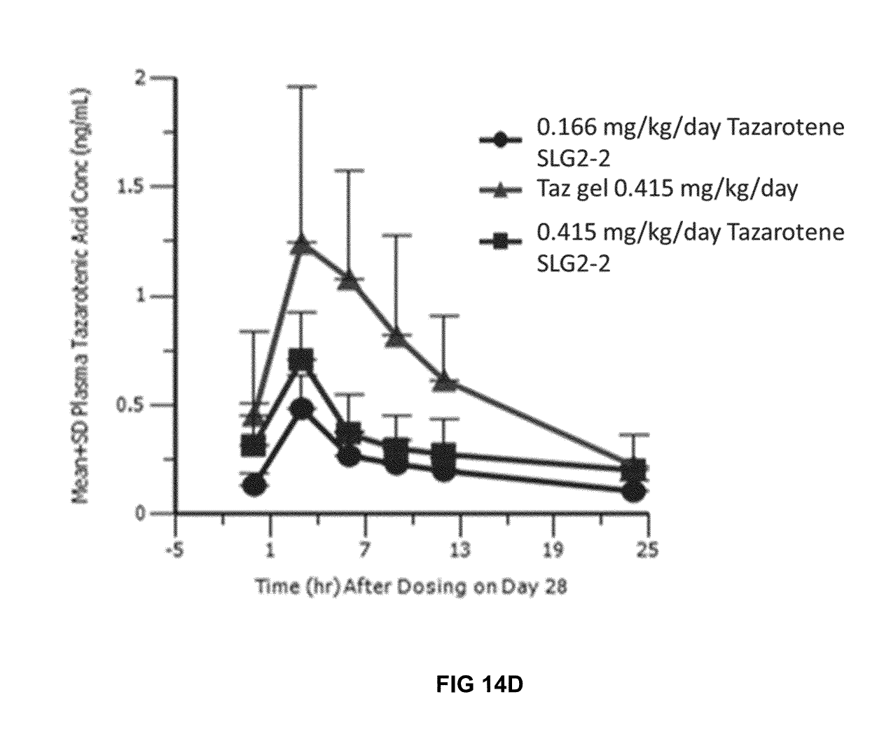

A further experiment were conducted to study the effects of high viscosity formulations on the efficacy the formulations. The formulations used to obtain the Table 21 had viscosities between about 150 to about −200 pa·s at room temperature (about 20-23 or 20-25 degrees C.), while other formulations tested has viscosities above about 800 pa·s at room temperature. The particle size of the solid lipid nanoparticle (SLN), after preparation, was less than 10 μm. The difference between SLG2 and SLG2-2 lied in different compositions. SLG2-2 had BHA, BHT, and phenoxyethanol, while SLG2 did not have these components. FIG. 14 clearly showed that SLG2, SLG2-2, and SLG2-2 0.04% had a reduction of acinic area of about 30%, 25% and 43%, respectively. Sebaceous gland inhibition in hamster is significant for both concentrations of the tazarotene used, and the activity of SLG2-2 0.1% is low-moderate comparing to historical microsphere data. SLG2-2 showed biphasic curve and 0.04% formulation had better activity.

Surface irritation of SLG2 related formulations appears similar in the hamster model. The pig ear study was also performed using the SLG2-2 formulation (Table 20). 75% of all hair follicles showed SLN penetration, and the average deepest penetration can reach 445 μm. The Table 20, SLG2-4 formulation had additional 0.2% Tween 80. The use Tween 80 was added to improve the dispersion of SLNs in gels, and prevent the aggregation of lipid particles. The SLG2-4 formulation led to the delivery of SLNs into all the available hair follicles in the skin. These results showed that, despite the increased viscosity of SLG2-2, efficient hair follicle delivery and good efficacy were still achieved. Interestingly use of 0.04% API strength led to significantly improved efficacy.

### Dermal Tolerance in Minipig Model

An experiment with five of formulations MSG1, MSG2, MSC, SLG1, SLG2 in a one month mini pig tolerance study was carried out. The design for this study is shown by Table 22. In this study there were 5 groups of pigs, each dosed daily for 28 days with the three formulations shown for that group (the three formulations included one vehicle formulation and one known Tazaroc gel formulation).

TABLE 22
Design of the 28 day Minipig Study

| | | | | | Number of |
|---|---|---|---|---|---|
| | | Dose Level | Formulation | Test Article | Animals |
| Group | | % (w/w) | Dose | Dose | Main Study |
| No. | Test Material | (mg/kg/day) | (mg/cm²) | (mg/cm²) | Males |
| 1 | Vehicle for | 0 | 22 | 0 | 3 |
| | Taz MSG1 | | | | |
| | Taz Gel | 0.1 | 22 | 0.022 | |
| | Taz MSG1 | 0.1 | 22 | 0.022 | |
| 2 | Vehicle for | 0 | 22 | 0 | 3 |
| | Taz MSG2 | | | | |
| | Taz Gel | 0.1 | 22 | 0.022 | |
| | Taz MSG2 | 0.1 | 22 | 0.022 | |
| 3 | Vehicle for | 0 | 22 | 0 | 3 |
| | Taz MSC | | | | |
| | Taz Gel | 0.1 | 22 | 0.022 | |
| | Taz MSC | 0.1 | 22 | 0.022 | |
| 4 | Vehicle for | 0 | 22 | 0 | 3 |
| | Taz SLG 1 | | | | |
| | Taz Gel | 0.1 | 22 | 0.022 | |
| | Taz SLG 1 | 0.1 | 22 | 0.022 | |
| 5 | Vehicle for | 0 | 22 | 0 | 3 |
| | Taz SLG 2 | | | | |
| | Taz Gel | 0.1 | 22 | 0.022 | |
| | Taz SLG 2 | 0.1 | 22 | 0.022 | |

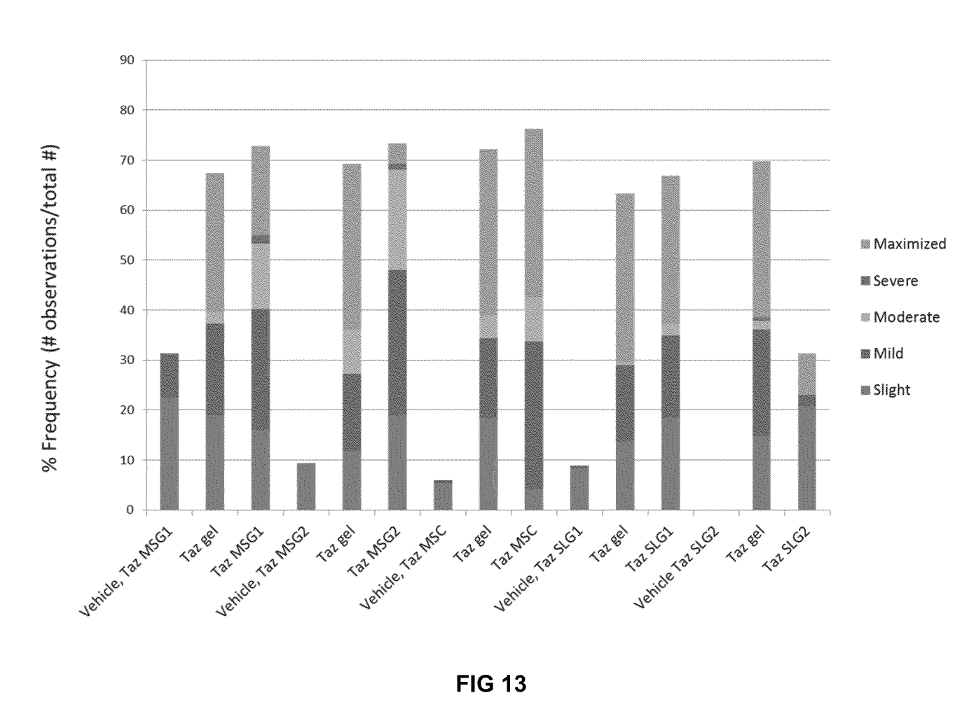

As shown in FIG. 13, all vehicle only formulations in this minipig study were no tazarotene formulations that well tolerated and showed very low frequency and low intensity of erythema. These include the vehicles from or for the MSG1, MSG2, MSC, SLG1 and SLG2 formulations. Among these vehicles, the MSG1 vehicle showed the highest frequency and severity, while no sign of erythema was found for SLG2 vehicle. The use of 60% glycerin in MSG1, in contrast to 30% glycerin and 30% PEG 400 in MSG2, may cause the difference in the tolerance between MSG1 and MSG2 vehicles. TAZ MSG1, MSG2, MSC and SLG1 showed significantly increased erythema compared to their respective vehicles. Among them, MSC seemed to have higher incidence of mild and maximized erythema, and lowest incidence of slight erythema, followed by MSG1. MSG2 formulations, like MSG2 vehicles, showed reduced frequency of maximized erythema and higher frequency of slight erythema, compared to MSG1. For TAZ SLG2, highest frequency of animals was found to be absent from erythema (about 70%), 20% of animals were found to have slight erythema, while less 10% was found to have maximized level of erythema. As a control, TAZ gel showed similar erythema across the groups. These results showed that SLG2 seemed to be the best formulation with improved tolerance in mini pigs. Ranking of test formulations for erythema TazSLG2<<TazMSG2<Taz MSG1<TazSLG1<TazMSC˜Taz gel (˜ means about equal to).

The TazSLG2 formulation proved to be the best formulation in the mini pig tolerance study as shown by FIG. 13. Another tolerance and PK study was performed with the SLG2-2 (0.1% Taz) and SLG2-9 (0.04% Taz). Data were plotted to show the estimated scores in each animals, as shown in FIG. 14. It is evident that SLG2-2 vehicles are well tolerated without any sign of erythema, eschar and edema. Compared to the Tazorac® Gel, 0.1% TAZ SLG2-2 formulation modestly improved the tolerance in terms of erythema, eschar and edema, while a lower strength of Taz (0.04%) SLG2-9 further improved the tolerance. Lower frequency and severity of effects were observed in both SLG2 formulations than Tazorac® Gel. 0.1% SLG2 and 0.04% SLG2 were also found to reduce the systemic exposure of Taz in blood systems, further confirmed the safety of the SLG2 formulations.

A number of formulations have been evaluated based on in vivo tolerability, in vivo efficacy, chemical and physical stability, antimicrobial activity, manufacturability. The TazSLG2 formulations with a strength of 0.1% and 0.04% are desirable tazarotene encapsulated into SLN as SLG formulations.

As set forth supra a preferred TazSLG dermal compositions comprise TazSLN in a gel vehicle. A gel is a semisolid to solid, jelly like material that is a substantially dilute cross linked system that exhibits essentially no flow when in the steady-state. the dermal compositions can, instead of a gel vehicle, alternately comprise a viscous liquid carrier, such as lotion, which comprises a lower molecular weight polymer as compared to a gel. By weight a gel is mostly liquid but behaves like a solid due to a three-dimensional cross-linked network within the liquid. Thus a gel is a non-fluid colloidal network or polymer network that is expanded throughout its whole volume by a fluid (such as water, in the case of a hydrogel).

As noted supra a preferred TazSLG formulation comprises a gel made from a carbopol (a carbopol is also known as a carbomer). A carbomer is a homopolymer of acrylic acid cross linked with a polyalcohol allyl ether. A desirable TazSLG formulation comprises a gel made using about 0.35 wt % to about 0.5 wt % carbopol, 0.05% EDTA (ethylenediaminetetraacetic acid, as chelating agent), 0.1% sodium thiosulfate, 0.05% BHT, 0.05% BHT, 2% solutol HS 15, 0.2% methylparaben, 0.1% propylparaben, and 1% phenoxyethanol and is a desirable formulation because such a formulation exhibited, when topically administered, a 40% reduction of sebum production by follicular sebaceous gland. For detailed TazSLG formulations see e.g. Tables 11, 12 and 19 in the patent application. The solid lipid nanoparticles can preferably comprise 7% glyceryl behenate (compritol 888 ATO).

A desirable carbomer in the TazSLG formulation is Carbopol 974P available from Lubrizol Advanced Materials, Inc. Carbopol 974P is a polymer of carboxypolymethylene and is a carbomer, specifically a carbomer homopolymer type B. Carbopol 974P has a viscosity, cP at 25 degrees C. of between about 29,400 to about 39,400, as determined by the Brookfield RVT method at 20 rpm, neutralized to pH 7.3 to 7.8. preferably residual monomer (i.e. free acrylic acid) is less than about 1,000 ppm.

In addition, the process for making gel vehicle component of the TazSLG described herein uses a gelling agent. The gelling agent can be, for example, acacia, alginic acid, bentonite, Carbopols® (also known as carbomers), carboxymethylcellulose. ethylcellulose, gelatin, hydroxyethylcellulose, hydroxypropyl cellulose, magnesium aluminum silicate (Veegum®), methylcellulose, poloxamers (Pluronics®), polyvinyl alcohol, sodium alginate, tragacanth, and xanthan gum. Though each gelling agent has some unique properties, there are some generalizations that can be made. For example, carbomers require a pH adjustment to create the gel after the gelling agent has been wetted in the dispersing medium. Carbomers comprise a family of Carbopol polymers. Generally used as a dry powder with a high bulk density that forms an acidic aqueous solutions (pH around 3.0) which thicken at higher pHs (around 5 or 6). Carbomers swell in aqueous solution at that pH as much as 1000 times their original volume. Their solutions range in viscosity from 0 to 80,000 centipoise (cps). Examples of carbopol gelling agents and their viscosities (within parentheses) in a 0.5% solution at pH 7.5 and at room temperature are: Carbopol® 910 (3,000-7,000), Carbopol® 934 (30,500-39,400), Carbopol® 934P (29,400-39,400), Carbopol® 940 (40,000-60,000), and Carbopol® 941 (4,000-11,000).

Patents and publications mentioned in the specification are indicative of the levels of those skilled in the art to which the invention pertains. These patents and publications are incorporated herein by reference to the same extent as if each individual application or publication was specifically and individually incorporated herein by reference.

While the invention has been described in terms of various specific and preferred embodiments, the skilled artisan will appreciate that various modifications, substitutions, omissions, and changes may be made without departing from the spirit thereof. In particular, the indication of a particular embodiment or parameter as being “preferred” should not be construed as indicating that other embodiments and/or parameters described herein are not desirable. Accordingly, it is intended that the scope of the present invention be limited solely by the scope of the following claims, including equivalents thereof.

We claim:

1. A composition for topical dermal administration comprising:
(a) tazarotene encapsulated by a lipid, and;
(b) a gel vehicle into which the lipid encapsulated retinoid is mixed or dispersed.

2. The composition of claim 1 wherein the lipid is a solid at room temperature.

3. The composition of claim 2 wherein the lipid has a melting point at about or greater than about 32° C.

4. The composition of claim 3 wherein the lipid is in the form of a plurality of biodegradable, solid lipid nanoparticles encompassing or encapsulating the tazarotene, thereby providing TazSLN and TazSLN in the gel vehicle provides TazSLG.

5. The composition of claim 4 further comprising a surfactant and wherein the gel is formed by a gelling agent.

6. The composition of claim 5 wherein the solid lipid nanoparticles have an average diameter no greater than about 5 μm.

7. The composition of claim 6 wherein the solid lipid nanoparticles have an average diameter no greater than about 3 μm.

8. The composition of claim 7, wherein the nanoparticles have an average diameter no greater than about 1 μm.

9. The composition of claim 6, wherein the lipid is selected from the group consisting of glyceryl dibehenate, glyceryl behenate myristyl myristate, myristyl laurate, triglycerides of C₁₀-C₂₂ fatty acids, propylene glycol monopalmiteostearate, cetyl palmitate, isostearyl isostearate, and propylene glycol monopalmiteostearate, and combinations thereof.

10. The composition of claim 9, wherein the lipid is myristyl myristate.

11. The composition of claim 9 wherein the lipid is glyceryl dibehenate or glyceryl dibehenate.

12. A method for treating a condition associated with excess sebum production, the method comprising topically applying to the skin of a patient in need thereof the composition of claim 1.

13. The method of claim 12, wherein the condition is selected from the group consisting of acne vulgaris, seborrhoeic dermatitis, psoriasis, and keratosis pilaris.

14. A composition for topical dermal administration, the composition comprising:
(a) tazarotene encapsulated by a plurality of biodegradable, solid lipid nanoparticles, thereby forming TazSLN, and;
(c) a polymeric gel vehicle into which the TazSLN is mixed or dispersed, thereby forming TazSLG,
wherein the lipid comprising solid the lipid nanoparticles is selected from the group consisting of glyceryl dibehenate, glyceryl behenate myristyl myristate, myristyl laurate, triglycerides of C₁₀-C₂₂ fatty acids, propylene glycol monopalmiteostearate, cetyl palmitate, isostearyl isostearate, and propylene glycol monopalmiteostearate, and combinations thereof; and
wherein the polymeric gel is comprises a gel or a gelling agent selected from the group consisting of a carbomer, acacia, alginic acid, bentonite, carboxymethylcellulose. ethylcellulose, gelatin, hydroxyethylcellulose, hydroxypropyl cellulose, magnesium aluminum silicate, methylcellulose, poloxamers, polyvinyl alcohol, sodium alginate, tragacanth, and xanthan gum.

15. A method for treating a condition associated with sebum production, the method comprising the step of topically applying to the skin of a patient in need of such treatment a dermal composition comprising:
(1) solid lipid nanoparticles, wherein the solid lipid nanoparticles comprise
a) a lipid selected from the group consisting of glyceryl dibehenate, glyceryl behenate myristyl myristate, myristyl laurate, triglycerides of C₁₀-C₂₂ fatty acids, propylene glycol monopalmiteostearate, cetyl palmitate, isostearyl isostearate, and propylene glycol monopalmiteostearate, and combinations thereof, and;
b) encapsulating by or encompassed by all or substantially all of the solid lipid nanoparticles is a compound of the formula: 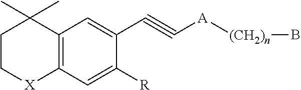
wherein:
X is S, O, or —N(R¹)— where R¹ is hydrogen or lower alkyl;
R is hydrogen or lower alkyl;
A is pyridinyl, thienyl, furyl, pyridazinyl, pyrimidinyl or pyrazinyl;
n is 0-2;
B is selected from the group consisting of: H, —COOH or a pharmaceutically acceptable salt, ester or amide of said —COOH group, —CH₂OH or an ether or ester derivative of said —CH₂OH group, —CHO or an acetal derivative of said —CHO group, and —COR² or a ketal derivative of said —COR² group, wherein R² is —(CH₂)ₘCH₃ wherein m is 0-4;
and;
(2) a polymeric gel as a carrier or as a vehicle for the solid lipid nanoparticles,
wherein the solid lipid nanoparticles have an average diameter between about 0.1 μm and about 10 μm; and wherein the compound penetrates the hair follicle to the depth of the sebaceous gland, and acts directly on the gland to reduce sebum production by the sebaceous gland, thereby treating the condition.

16. A method for treating a condition associated with excess sebum production, the method comprising the step of topically applying to the skin of a patient in need of such treatment a dermal composition comprising:
(1) solid lipid nanoparticles, wherein the solid lipid nanoparticles comprise
a) a lipid from the group consisting of glyceryl dibehenate, glyceryl behenate myristyl myristate, myristyl laurate, triglycerides of C₁₀-C₂₂ fatty acids, propylene glycol monopalmiteostearate, cetyl palmitate, isostearyl isostearate, and propylene glycol monopalmiteostearate, and combinations thereof, and;
b) encapsulating by or encompassed by all or by significantly all of the solid lipid nanoparticles a compound of the formula: 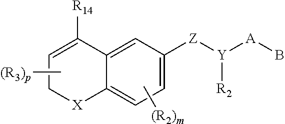
or a pharmaceutically acceptable salt thereof, wherein
X is S, O, NR′ where R′ is H or alkyl of 1 to 6 carbons, or X is [C(R₁)₂]ₙ where R₁ is independently H or alkyl of 1 to 6 carbons, and n is an integer between, and including, 0 and 2, and;
R₂ is hydrogen, lower alkyl of 1 to 6 carbons, F, Cl, Br, I, CF₃, fluoro substituted alkyl of 1 to 6 carbons, OH, SH, alkoxy of 1 to 6 carbons, or alkylthio of 1 to 6 carbons, and;
R₃ is hydrogen, lower alkyl of 1 to 6 carbons or F, and;
m is an integer having the value of 0-3, and;
p is an integer having the value of 0-3, and;
Z is —C≡C—, —N═N—, —N═CR₁—, —CR₁═N, —(CR₁═CR₁)_{n′}— where n′ is an integer having the value 0-5, —CO—NR₁—, —CS—NR₁—, —NR₁—CO, —NR₁—CS, —COO—, —OCO—, or —CSO—, —OCS—;
Y is a phenyl or naphthyl group, or heteroaryl selected from a group consisting of pyridyl, thienyl, furyl, pyridazinyl, pyrimidinyl, pyrazinyl, thiazolyl, oxazolyl, imidazolyl and pyrrazolyl, said phenyl and heteroaryl groups being optionally substituted with one or two R₂ groups, or, when Z is —(CR₁═CR₁)_{n′}— and n′ is 3, 4 or 5 then Y represents a direct valence bond between said (CR₂═CR₂)_{n′} group and B;
A is (CH₂)_{q} where q is 0-5, lower branched chain alkyl having 3-6 carbons, cycloalkyl having 3-6 carbons, alkenyl having 2-6 carbons and 1 or 2 double bonds, alkynyl having 2-6 carbons and 1 or 2 triple bonds;
B is hydrogen, COOH or a pharmaceutically acceptable salt thereof, COOR₈, CONR₉R₁₀, —CH₂OH, CH₂OR₁₁, CH₂OCOR₁₁, CHO, CH(OR₁₂)₂, CHOR₁₃O, —COR₇, CR₇(OR₁₂)₂, CR₇OR₁₃O, or tri-lower alkylsilyl, where R₇ is an alkyl, cycloalkyl or alkenyl group containing 1 to 5 carbons, R₈ is an alkyl group of 1 to 10 carbons or trimethylsilylalkyl where the alkyl group has 1 to 10 carbons, or a cycloalkyl group of 5 to 10 carbons, or R₈ is phenyl or lower alkylphenyl, R₉ and R₁₀ independently are hydrogen, an alkyl group of 1 to 10 carbons, or a cycloalkyl group of 5-10 carbons, or phenyl or lower alkylphenyl, R₁₁ is lower alkyl, phenyl or lower alkylphenyl, R₁₂ is lower alkyl, and R₁₃ is divalent alkyl radical of 2-5 carbons, and
R₁₄ is (R₁₅)ᵣ-phenyl, (R₁₅)ᵣ-naphthyl, or (R₁₅)ᵣ-heteroaryl where the heteroaryl group has 1 to 3 heteroatoms selected from the group consisting of O, S and N, r is an integer having the values of 0-5, and
R₁₅ is independently H, F, Cl, Br, I, NO₂, N(R₈)₂, N(R₈)COR₈, NR₈CON(R₈)₂, OH, OCOR₈, OR₈, CN, an alkyl group having 1 to 10 carbons, fluoro substituted alkyl group having 1 to 10 carbons, an alkenyl group having 1 to 10 carbons and 1 to 3 double bonds, alkynyl group having 1 to 10 carbons and 1 to 3 triple bonds, or a trialkylsilyl or trialkylsilyloxy group where the alkyl groups independently have 1 to 6 carbons;
and;
(2) a polymeric gel as a carrier or as a vehicle for the solid lipid nanoparticles,
wherein the solid lipid nanoparticles have an average diameter between about 0.1 μm and about 10 μm; and wherein the compound penetrates the hair follicle to the depth of the sebaceous gland, and acts directly on the gland to reduce the excess sebum production by the gland, thereby treating the condition.

17. A process for making TazSLG, the process comprising the steps of:
(a) preparing a lipid phase by making a hot melt homogenization of a lipid by heating a mixture of a lipid and a retinoid to a temperature of between about 75° C. and about 90° C.;
(b) preparing an aqueous phase comprising water and a surfactant, also heated to a temperature of between about 75° C. and about 90° C.;
(c) mixing together the lipid phase and the aqueous phase;
(d) cooling the step (c) mixture to between about 5° C. and about 15° C. over a period of time of about 5 minutes, thereby preparing a suspension in water of tazarotene encapsulated solid lipid particles (TazSLN);
(e) preparing a gel phase by adding a carbomer gelling agent to water and mixing;
(f) mixing together the gel phase and the TazSLN suspension, and adjust the pH of the mixture to between about pH 5.5 to about pH 6.0, thereby making TazSLG.

18. Solid lipid particles encapsulating tazarotene and in a gel vehicle (TazSLG) made by the process of claim 17.

19. A composition for topical dermal administration comprising:
(a) a retinoid encapsulated by a fatty acid ester, and;
(b) a gel vehicle into which the fatty acid ester encapsulated retinoid is mixed or dispersed.

20. A composition for topical dermal administration comprising:
(a) a population of solid lipid nanoparticles comprising:
(i) butylated hydroxytolune;
(ii) butylated hydroxyanisole;
(iii) a surfactant;
(iv) tazarotene, and;
(v) glyceryl behenate, and;
(b) a gel comprising:
(i) ethylenediaminetetraacetic acid;
(ii) a carboxypolymethylene carbomer homopolymer
(iii) sodium thiosulfate;
(iv) methylparaben;
(v) propylparaben;
(vi) phenoxyethanol, and;
(vii) sodium thiosulfate,
wherein the solid lipid nanoparticles have with an average particle size of between about 1 micron and about 10 microns, the solid lipid nanoparticles encapsulate or encompass substantially all the tazarotene, and the solid lipid nanoparticles are mixed into or dispersed within the gel, the gel serving as a vehicle for the tazarotene encapsulating or encompassing solid lipid nanoparticles.
